(19) United States
(12) Patent Application Publication
(10) Pub. No.: US 2024/0239791 A1
(43) Pub. Date: Jul. 18, 2024

(54) PROCESSES FOR THE SYNTHESIS OF VALBENAZINE

(71) Applicant: Neurocrine Biosciences, Inc., San Diego, CA (US)

(72) Inventor: John Lloyd Tucker, San Diego, CA (US)

(21) Appl. No.: 18/288,311

(22) PCT Filed: Apr. 25, 2022
(86) PCT No.: PCT/US2022/026208

§ 371 Date: Oct. 25, 2023

Related U.S. Application Data
(60) Provisional application No. 63/286,764, filed on Dec. 7, 2021.
(60) Provisional application No. 63/180,043, filed on Apr. 26, 2021.

(51) Int. Cl. C07D 471/04; A61K 9/48; A61K 9/50; A61K 31/4375

(52) U.S. Cl. CPC C07D 471/04; A61K 9/4866; A61K 9/5015; A61K 31/4375

(57) ABSTRACT

The present application relates to processes for preparing (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate), which is an inhibitor of vesicular monoamine transporter 2 (VMAT2) useful in the treatment of hyperkinetic movement disorders such as tardive dyskinesia (TD).

### FIELD OF THE INVENTION

The present application relates to processes for preparing (S)-(2R,3R, 11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate), which is an inhibitor of vesicular monoamine transporter 2 (VMAT2) useful in the treatment of hyperkinetic movement disorders such as tardive dyskinesia (TD).

### BACKGROUND OF THE INVENTION

Hyperkinetic disorders are characterized by excessive, abnormal involuntary movement. These neurologic disorders include tremor, dystonia, ballism, tics, akathisia, stereotypies, chorea, myoclonus, and athetosis. Though the pathophysiology of these movement disorders is poorly understood, it is thought that dysregulation of neurotransmitters in the basal ganglia plays an important role (Kenney et al., Expert Review Neurotherapeutics, 2005, 6, 7-17). The chronic use and high dosing of typical neuroleptics or centrally acting dopamine receptor blocking antiemetics predispose patients to the onset of tardive syndromes. Tardive dyskinesia, one subtype of the latter syndromes, is characterized by rapid, repetitive, stereotypic, involuntary movements of the face, limbs, or trunk (Muller, Expert Opin. Investig. Drugs, 2015, 24, 737-742).

The reversible inhibition of the vesicular monoamine transporter-2 system (VMAT2) by 3-isobutyl-9,10-dimethoxy-1,3,4,6,7,11b-hexahydro-2H-pyrido[2,1-a]isoquinolin-2-one, also known as tetrabenazine (TBZ), improves the treatment of various hyperkinetic movement disorders. However, the draw backs to such treatment are the fluctuating response, the need for frequent intake due to TBZ rapid metabolism, and side effects. Side effects associated with TBZ include sedation, depression, akathisia, and parkinsonism.

Tetrabenazine, contains two chiral centers and is a racemic mixture of two stereoisomers, is rapidly and extensively metabolized in vivo to its reduced form, 3-isobutyl-9, 10-dimethoxy-1,3,4,6,7,11b-hexahydro-2H-pyrido[2,1-a]isoquinolin-2-ol, also known as dihydrotetrabenazine (DHTBZ). DHTBZ is thought to exist as four individual isomers: (±) alpha-DHTBZ and (±) beta-DHTBZ. The (2R,3R, 11bR) or (+) alpha-DHTBZ is reported to be the absolute configuration of the active metabolite (Kilbourn et al., Chirality, 1997, 9, 59-62). Tetrabenazine has orphan drug status in the US and is approved in certain European countries. Its use is also allowed for therapy of chorea in patients with Huntington's disease. However, tetrabenazine is rapidly metabolized and must frequently be administered throughout the day (Muller, Expert Opin. Investig. Drugs, 2015, 24, 737-742).

Ingrezza®, the first FDA approved therapy for patients suffering from tardive dyskinesia, contains Valbenazine [(S)-(2R,3R, 11bR)-3-isobutyl-9,10-dimethoxy-2,3,4,6,7, 1 1b-hexahydro-1H-pyrido[2,1-a]isoquinolin-2-yl 2-amino-3-methylbutanoate], and is present as Valbenazine ditosylate. Valbenazine is a potent and selective VMAT2 inhibitor and is a prodrug of the (+)-α-isomer of dihydrotetrabenazine. The (+)-α-isomer of dihydrotetrabenazine is reported to be the most potent isomer of the dihydrotetrabenazine isomers (binding affinity, Kᵢ=0.97 nM; and absolute configurations reported by Kilbourn et al., Chirality, 1997, 9, 59-62). Valbenazine ditosylate is referred to herein as the compound of Formula I.

Methods for synthesizing (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate have been described in, for example, WO2008/058261, WO2017/112857, and WO2021/050977, each of which is incorporated herein by reference in its entirety. Certain salts and crystal forms for Valbenazine have been described in WO2017/075340, and certain formulations for Valbenazine have also been described in WO2019/060322, each of which is incorporated herein by reference in its entirety.

Due to the high demand for and the usefulness of Ingrezza®, there is a need for the development of new processes for its manufacture, particularly more environmentally friendly processes. This application is directed towards this need and others.

### SUMMARY OF THE INVENTION

The present invention is directed, inter alia, to processes useful in the preparation of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (compound of Formula I), intermediates, and crystalline forms related thereto.

One aspect of the present invention encompasses, inter alia, certain processes for preparing a compound of Formula I:

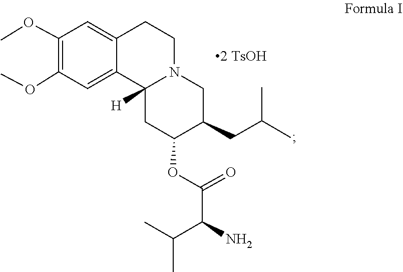 [SMILES: COc1cc2c(cc1OC)[C@H]1C[C@@H](OC(=O)[C@@H](N)C(C)C)[C@H](CC(C)C)CN1CC2]

comprising the steps of:
a) reacting a compound of Formula F1:

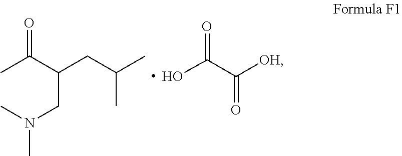

with a Step a)-base to afford a compound of Formula F2:

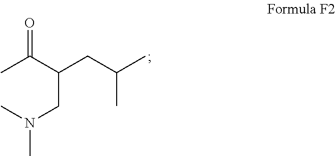

b) cyclizing the compound of Formula F2 with a compound of Formula F3:

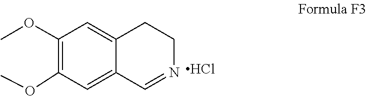

in the presence of sodium iodide to afford a compound of Formula F4:

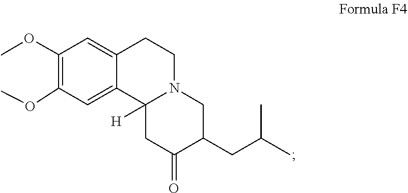 [SMILES: COc1cc2c(cc1OC)C1CC(=O)C(CC(C)C)CN1CC2]

c) reducing the compound of Formula F4 with a reducing agent to afford a compound of Formula F5:

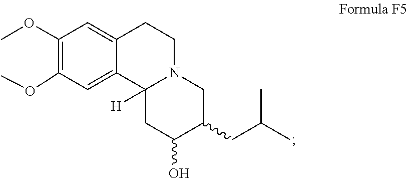 [SMILES: COc1cc2c(cc1OC)C1CC(O)C(CC(C)C)CN1CC2]

d) resolving the compound of Formula F5 with (S)-(+)-camphorsulfonic acid (CSA) to afford a compound of Formula F6-CSA:

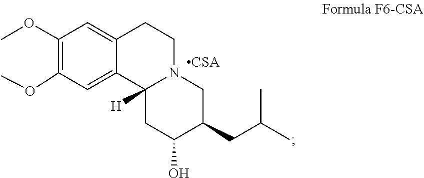 [SMILES: COc1cc2c(cc1OC)[C@H]1C[C@@H](O)[C@H](CC(C)C)CN1CC2]

e) reacting the compound of Formula F6-CSA with a Step e)-base to afford a compound of Formula F6:

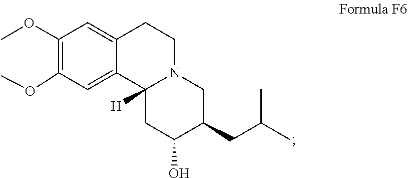 [SMILES: COc1cc2c(cc1OC)[C@H]1C[C@@H](O)[C@H](CC(C)C)CN1CC2]

f) coupling the compound of Formula F6 and a carboxylic acid of Formula F7:

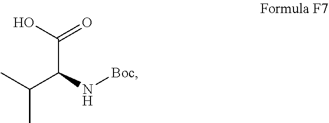

with a coupling reagent to afford a compound of Formula F8:

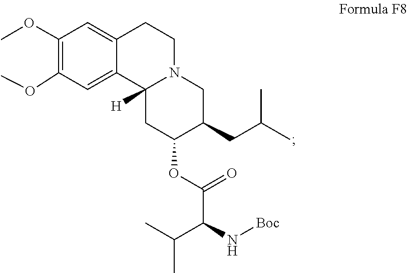 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) deprotecting the compound of Formula F8 with hydrogen chloride to afford a compound of Formula F9-HCI:

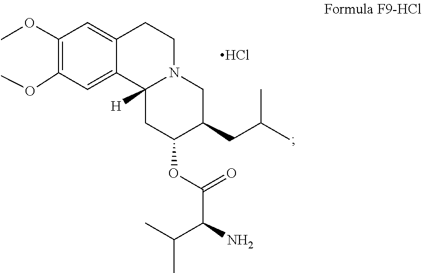 [SMILES: COc1cc2c(cc1OC)[C@H]1C[C@@H](OC(=O)[C@@H](N)C(C)C)[C@H](CC(C)C)CN1CC2 ; Cl]

h) reacting the compound of Formula F9-HCI with a Step h)-base to afford a compound of Formula F9 (free base):

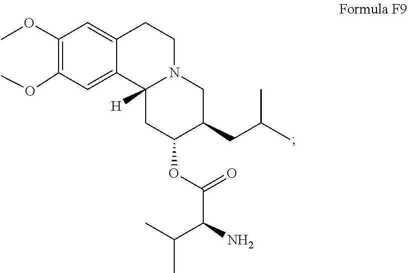 [SMILES: COc1cc2c(cc1OC)[C@H]1C[C@@H](OC(=O)[C@@H](N)C(C)C)[C@H](CC(C)C)CN1CC2]

and
i) reacting the compound of Formula F9 with p-toluenesulfonic acid to afford the compound of Formula I.

Another aspect of the present application provides processes for preparing pharmaceutical compositions comprising: preparing a compound of Formula I according to any of the processes described herein; and formulating the compound of Formula I with a pharmaceutically acceptable carrier and/or diluent.

Another aspect of the present application provides processes for preparing unit dosage forms comprising: preparing a compound of Formula I according to any of the processes described herein; and formulating the compound of Formula I with a pharmaceutically acceptable carrier and/or diluent.

In some embodiments, the compound of Formula I is crystalline. In some embodiments, the compound of Formula I is crystalline Form I, crystalline Form II, crystalline Form III, crystalline Form IV, crystalline Form V, crystalline Form VI, or an amorphous solid as described in WO2017/075340, incorporated herein by reference in its entirety. In some embodiments, the crystalline compound of Formula I is Form I.

Another aspect of the present application provides pharmaceutical compositions prepared by any of the processes as described herein.

Another aspect of the present application provides unit dosage forms prepared by any of the processes as described herein.

Another aspect of the present application provides methods for inhibiting monoamine transporter isoform 2 (VMAT2) in a patient in need thereof, comprising administering to the patient a therapeutically effective amount of a pharmaceutical composition or a unit dosage form, wherein the pharmaceutical composition and the unit dosage form can be prepared according to any of the processes as described herein.

Another aspect of the present application provides methods of treating a neurological or psychiatric disease or disorder in a patient in need thereof, comprising administering to the patient a therapeutically effective amount of a pharmaceutical composition or a unit dosage form, wherein the pharmaceutical composition and the unit dosage form can be prepared according to any of the processes as described herein.

Another aspect of the present application provides methods of treating a hyperkinetic disorder in a patient in need thereof, comprising administering to the patient a therapeutically effective amount of a pharmaceutical composition or a unit dosage form, wherein the pharmaceutical composition and the unit dosage form can be prepared according to any of the processes as described herein.

Another aspect of the present application provides uses of a pharmaceutical composition or a unit dosage form for the manufacture of a medicament for inhibiting monoamine transporter isoform 2 (VMAT2) in a patient in need thereof, wherein the pharmaceutical composition and the unit dosage form can be prepared according to any of the processes as described herein.

Another aspect of the present application provides uses of a pharmaceutical composition or a unit dosage form for the manufacture of a medicament for treating a neurological or psychiatric disease or disorder in a patient in need thereof, wherein the pharmaceutical composition and the unit dosage form can be prepared according to any of the processes as described herein.

Another aspect of the present application provides uses of a pharmaceutical composition or a unit dosage form for the manufacture of a medicament for treating a hyperkinetic disorder in a patient in need thereof, wherein the pharmaceutical composition and the unit dosage form can be prepared according to any of the processes as described herein.

These and other aspects of the invention disclosed herein will be set forth in greater detail as the patent disclosure proceeds.

### BRIEF DESCRIPTION OF THE FIGURES

FIG. 1 shows a general synthetic scheme for the preparation of (2R,3R,11bR)-3-isobutyl-9,10-dimethoxy-2,3,4,6,7,11b-hexahydro-1H-pyrido[2,1-a]isoquinolin-2-ol (S)-(+)-camphorsulfonate (Compound of Formula F6-CSA) from 3-((dimethylamino)methyl)-5-methylhexan-2-one oxalate (Compound of Formula F1).

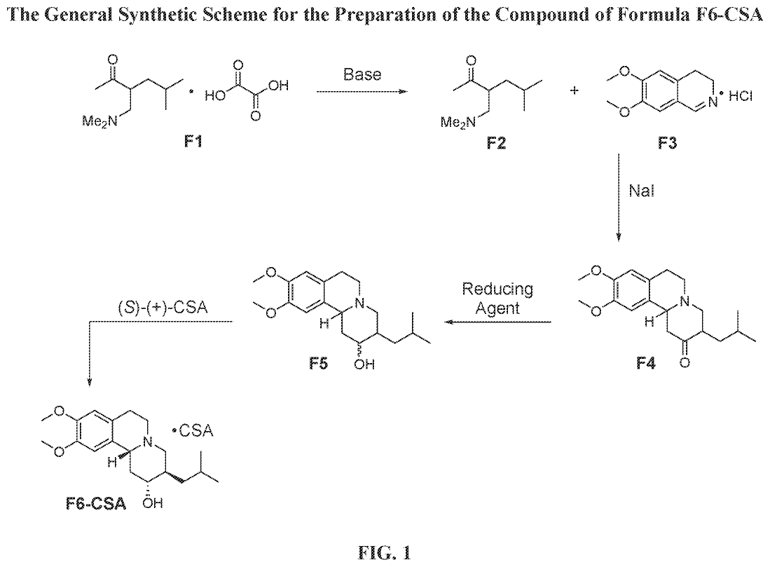

FIG. 2 shows a general synthetic scheme for the preparation of (S)-(2R,3R,11bR)-3-isobutyl-9, 10-dimethoxy-2,3,4,6,7,11b-hexahydro-1H-pyrido[2,1-a]isoquinolin-2-yl 2-amino-3-methylbutanoate di(4-methylbenzenesulfonate) (Compound of Formula I) from (2R,3R,11bR)-3-isobutyl-9, 10-dimethoxy-2,3,4,6,7, 1 1b-hexahydro-1H-pyrido[2, 1-a ]isoquinolin-2-ol (S)-(+)-camphorsulfonate (Compound of Formula F6-CSA), wherein the Compound of Formula F6-CSA can be prepared according to any of the methods as described herein.

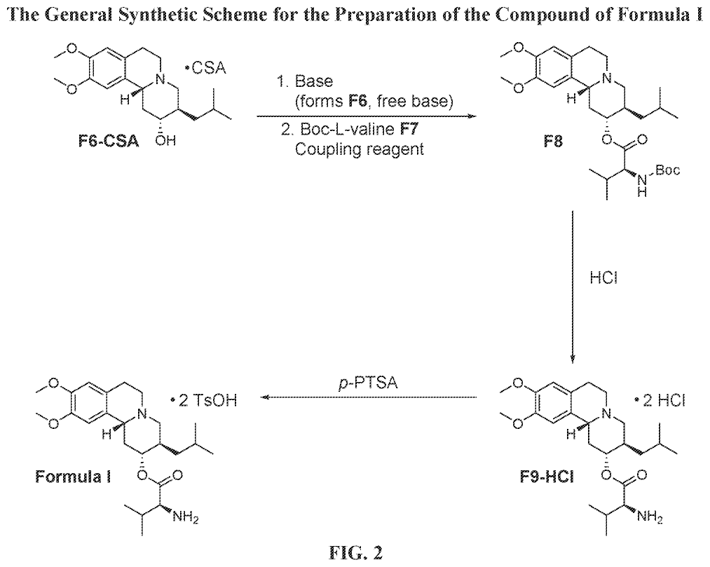

FIG. 3 shows a general synthetic scheme for the preparation of (2R,3R,11bR)-3-isobutyl-9,10-dimethoxy-2,3,4,6,7,11b-hexahydro-1H-pyrido[2,1-a]isoquinolin-2-ol (S)-(+)-camphorsulfonate (Compound of Formula F6-CSA) from 3-((dimethylamino)methyl)-5-methylhexan-2-one oxalate (Compound of Formula F1).

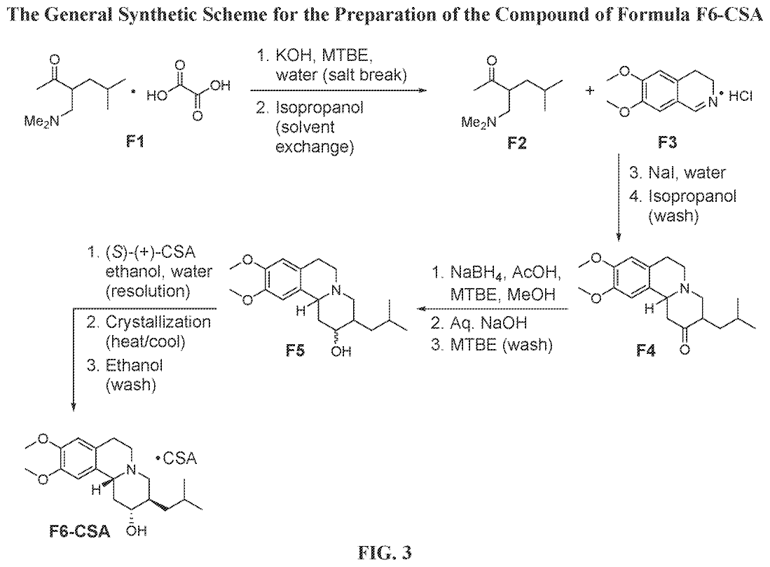

FIG. 4 shows a general synthetic scheme for the preparation of (S)-(2R,3R,11bR)-3-isobutyl-9, 10-dimethoxy-2,3,4,6,7,11b-hexahydro-1H-pyrido[2,1-a]isoquinolin-2-yl 2-amino-3-methylbutanoate di(4-methylbenzenesulfonate) (Compound of Formula I) from (2R,3R,11bR)-3-isobutyl-9, 10-dimethoxy-2,3,4,6,7,11b-hexahydro-1H-pyrido[2,1-a]isoquinolin-2-ol (S)-(+)-camphorsulfonate (Compound of Formula F6-CSA), wherein the Compound of Formula F6-CSA can be prepared according to any of the methods as described herein.

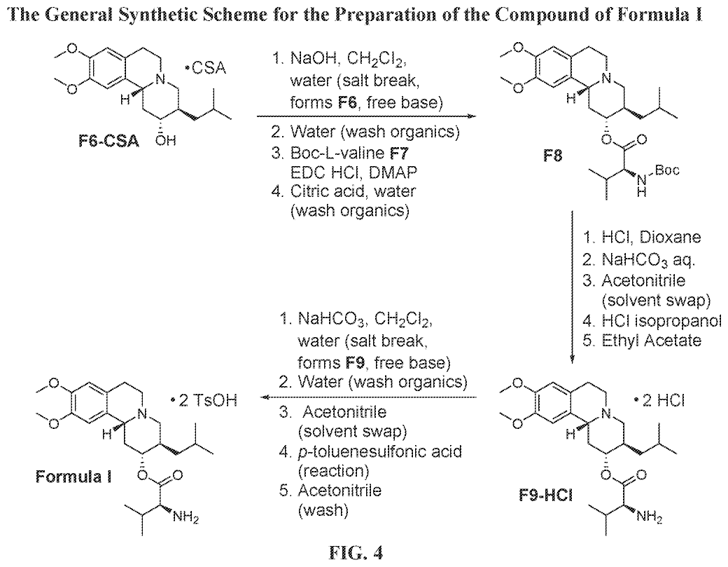

FIG. 5 shows a general synthetic scheme for the preparation of (S)-(2R,3R,11bR)-3-isobutyl-9, 10-dimethoxy-2,3,4,6,7,11b-hexahydro-1H-pyrido[2,1-a]isoquinolin-2-yl 2-amino-3-methylbutanoate di(4-methylbenzenesulfonate) (Compound of Formula I) from (2R,3R,11bR)-3-isobutyl-9, 10-dimethoxy-2,3,4,6,7,11b-hexahydro-1H-pyrido[2,1-a]isoquinolin-2-ol (S)-(+)-camphorsulfonate (Compound of Formula F6-CSA), wherein the Compound of Formula F6-CSA can be prepared according to any of the methods as described herein.

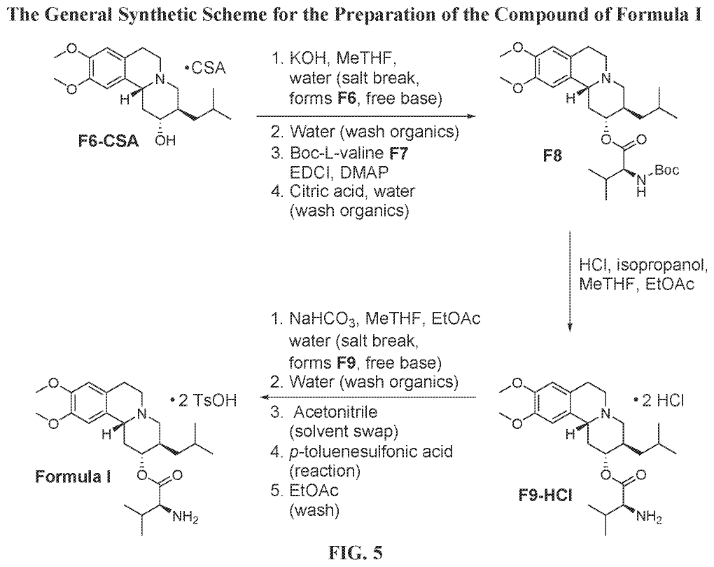

FIG. 6 shows a general synthetic scheme for the preparation of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Compound of Formula I) from (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate dihydrochloride (Compound of Formula F9-HCl), wherein the Compound of Formula F9-HCl can be prepared according to any of the methods as described herein.

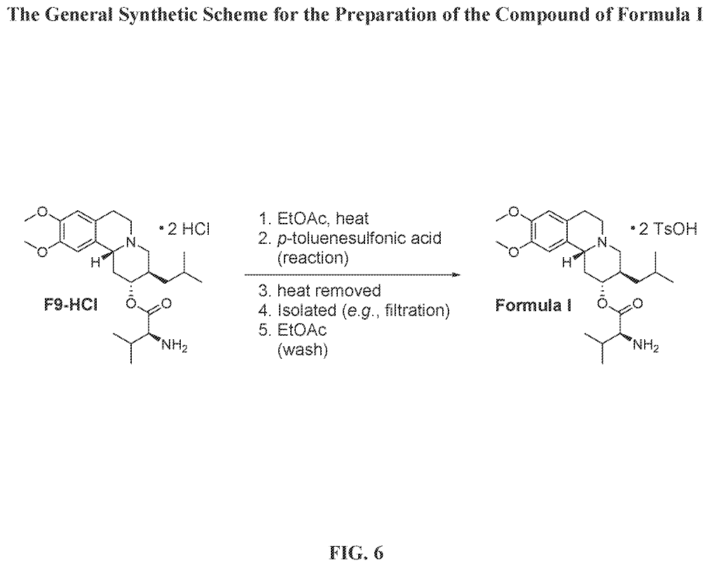

### DETAILED DESCRIPTION OF THE INVENTION

One aspect of the present invention provides, inter alia, certain processes for preparing a compound of Formula I:

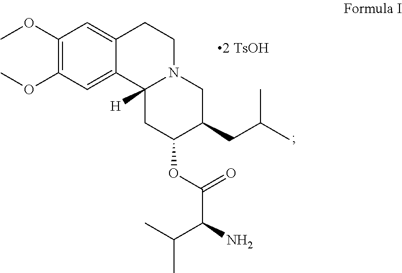 [SMILES: COc1cc2c(cc1OC)[C@H]1C[C@@H](OC(=O)[C@@H](N)C(C)C)[C@H](CC(C)C)CN1CC2]

comprising the steps of:
a) reacting a compound of Formula F1:

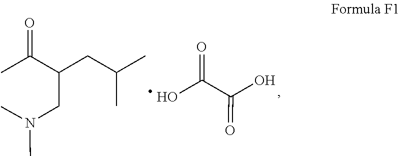

with a Step a)-base to afford a compound of Formula F2:

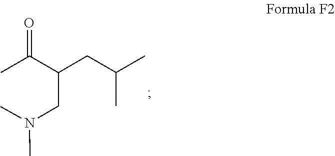

b) cyclizing the compound of Formula F2 with a compound of Formula F3:

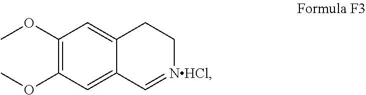

in the presence of sodium iodide to afford a compound of Formula F4:

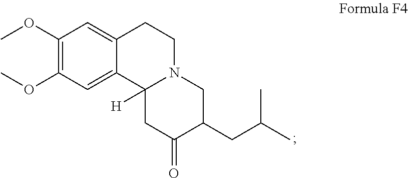 [SMILES: COc1cc2c(cc1OC)C1CC(=O)C(CC(C)C)CN1CC2]

c) reducing the compound of Formula F4 with a reducing agent to afford a compound of Formula F5:

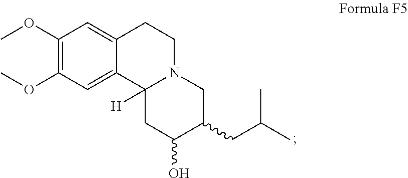 [SMILES: COc1cc2c(cc1OC)C1CC(O)C(CC(C)C)CN1CC2]

d) resolving the compound of Formula F5 with (S)-(+)-camphorsulfonic acid (CSA) to afford a compound of Formula F6-CSA:

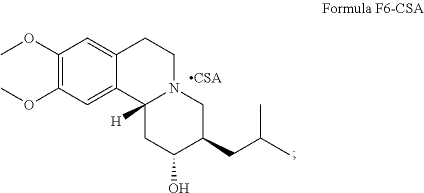 [SMILES: COc1cc2c(cc1OC)[C@H]1C[C@@H](O)[C@H](CC(C)C)CN1CC2]

e) reacting the compound of Formula F6-CSA with a Step e)-base to afford a compound of Formula F6:

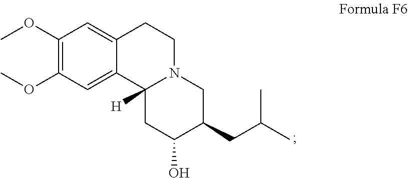 [SMILES: COc1cc2c(cc1OC)[C@H]1C[C@@H](O)[C@H](CC(C)C)CN1CC2]

f) coupling the compound of Formula F6 and a carboxylic acid of Formula F7:

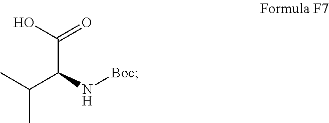

with a coupling reagent to afford a compound of Formula F8:

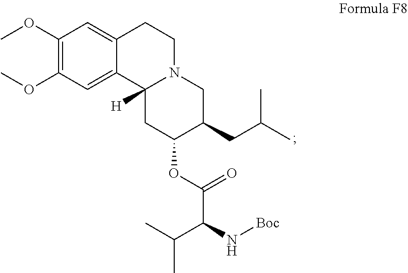 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) deprotecting the compound of Formula F8 with hydrogen chloride to afford a compound of Formula F9-HCl:

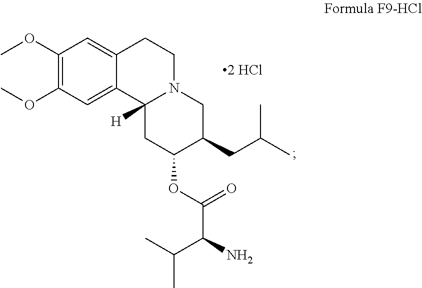 [SMILES: COc1cc2c(cc1OC)[C@H]1C[C@@H](OC(=O)[C@@H](N)C(C)C)[C@H](CC(C)C)CN1CC2]

h) reacting the compound of Formula F9-HCl with a Step h)-base to afford a compound of Formula F9 (free base):

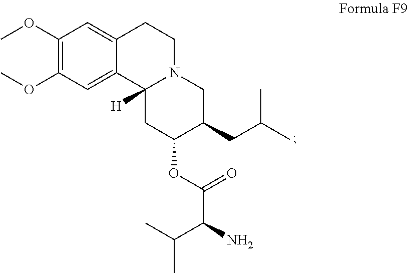 [SMILES: COc1cc2c(cc1OC)[C@H]1C[C@@H](OC(=O)[C@@H](N)C(C)C)[C@H](CC(C)C)CN1CC2]

and
i) reacting the compound of Formula F9 with p-toluenesulfonic acid to afford the compound of Formula I.

### Step a)—Processes for Preparing 3-((dimethylamino)methyl)-5-methylhexan-2-one (Compound of Formula F2).

In some embodiments, the compound of Formula F2 is prepared by the processes described herein comprising reacting a compound of Formula F1 with a Step a)-base to afford the compound of Formula F2:

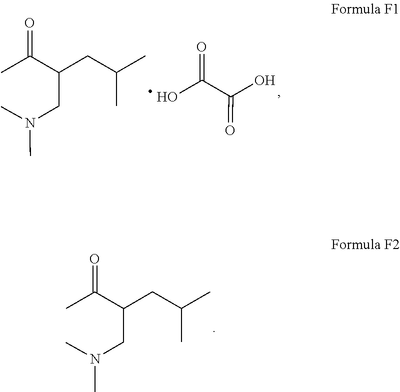

In some embodiments, reacting the compound of Formula F1 with a Step a)-base is carried out in the presence of a Step a)-solvent. The Step a)-solvent can be any suitable solvent. In some embodiments, reacting the compound of Formula F1 with a Step a)-base is carried out in the presence of a Step a)-solvent comprising methyl tert-butyl ether (MTBE). In some embodiments, reacting the compound of Formula F1 with a Step a)-base is carried out in the presence of methyl tert-butyl ether (MTBE).

In some embodiments, the Step a)-solvent is a mixture of solvents. In some embodiments, reacting the compound of Formula F1 with a Step a)-base is carried out in the presence of a Step a)-solvent comprising water and an organic solvent. In some embodiments, the mixture of solvents comprises water and an ether solvent. In some embodiments, reacting the compound of Formula F1 with a Step a)-base is carried out in the presence of a Step a)-solvent comprising water and methyl tert-butyl ether (MTBE).

In some embodiments, prior to the reacting with Step a)-base, the volume ratio of water to MTBE is from about 1:1 to about 4:1. In some embodiments, prior to the reacting with Step a)-base, the volume ratio of water to MTBE is from about 1.3:1 to about 3.5:1. In some embodiments, prior to the reacting with Step a)-base, the volume ratio of water to MTBE is from about 1.8:1 to about 3:1. In some embodiments, prior to the reacting with Step a)-base, the volume ratio of water to MTBE is from about 2.0:1 to about 2.8:1. In some embodiments, prior to the reacting with Step a)-base, the volume ratio of water to MTBE is from about 2.3:1 to about 2.5:1. In some embodiments, prior to the reacting with Step a)-base, the volume ratio of water to MTBE is from about 2.35:1 to about 2.45:1. In some embodiments, the volume ratio of water to MTBE is 2.4:1.

In some embodiments, the Step a)-base comprises an inorganic base. In some embodiments, the Step a)-base is a carbonate, hydrogen carbonate, or hydroxide base. In other embodiments, the Step a)-base is sodium carbonate. In some embodiments, the Step a)-base is potassium hydroxide. In some embodiments, the Step a)-base is aqueous potassium hydroxide. In some embodiments, the Step a)-base is an 8 wt % to 12 wt % potassium hydroxide solution. In some embodiments, the Step a)-base is a 10 wt % potassium hydroxide solution.

In some embodiments, reacting the compound of Formula F1 with a Step a)-base is carried out at a pH of about 10 to about 12. In some embodiments, reacting the compound of Formula F1 with a Step a)-base is carried out at a pH of about 11.

In some embodiments, the compound of Formula F2 is not isolated. Accordingly, in some embodiments, reacting the compound of Formula F1 with a Step a)-base is carried out in the presence of a Step a)-solvent and the Step a)-solvent is removed after completion of the reaction and replaced with a cyclizing-step solvent as described in Step b) (e.g., isopropanol (IPA)). In some embodiments, reacting the compound of Formula F1 with a Step a)-base is carried out in the presence of a Step a)-solvent, and after completion of the reaction (i.e., formation of the compound of Formula F2), the Step a)-solvent is removed and replaced with a cyclizing-step solvent as described in Step b) (e.g., a mixture of isopropanol (IPA) and water).

### Step b)—Processes for Preparing 3-isobutyl-9,10-dimethoxy-3,4,6,7-tetrahydro-1H-pyrido[2,1-a]isoquinolin-2(11b/)-one (Compound of Formula F4).

In some embodiments, the compound of Formula F4 is prepared by the processes described herein comprising cyclizing the compound of Formula F2 (for example, prepared as described in Step a)) with a compound of Formula F3 in the presence of sodium iodide to afford a compound of Formula F4:

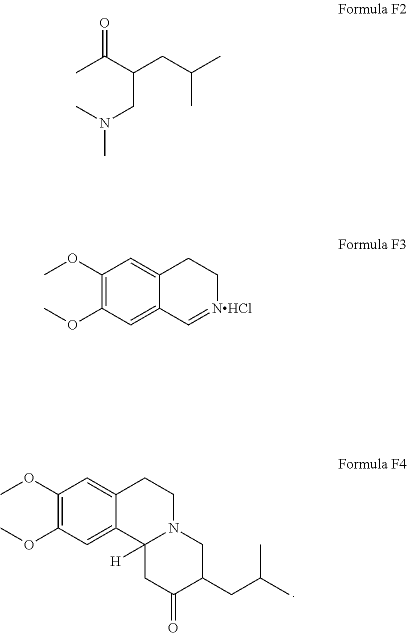

In some embodiments, the molar ratio of sodium iodide to the compound of Formula F3 is about 0.1:1 to 1:1. In some embodiments, the molar ratio of sodium iodide to the compound of Formula F3 is about 0.1:1 to 0.5:1. In some embodiments, the molar ratio of sodium iodide to the compound of Formula F3 is about 0.2:1 to 0.8:1. In some embodiments, the molar ratio of sodium iodide to the compound of Formula F3 is about 0.2: 1 to 0.6:1. In some embodiments, the molar ratio of sodium iodide to the compound of Formula F3 is about 0.25:1 to 0.55:1. In some embodiments, the molar ratio of sodium iodide to the compound of Formula F3 is about 0.3:1 to 0.5:1. In some embodiments, the molar ratio of sodium iodide to the compound of Formula F3 is about 0.35:1 to 0.45:1. In some embodiments, the molar ratio of sodium iodide to the compound of Formula F3 is about 0.4:1.

In some embodiments, cyclizing the compound of Formula F2 with a compound of Formula F3 in the presence of sodium iodide in Step b) is carried out in a cyclizing-step solvent. The cyclizing-step solvent can be any suitable solvent. In some embodiments, cyclizing the compound of Formula F2 with a compound of Formula F3 in the presence of sodium iodide in Step b) is carried out in a cyclizing-step solvent comprising isopropanol (IPA) and water. In some embodiments, cyclizing the compound of Formula F2 with a compound of Formula F3 in the presence of sodium iodide in Step b) is carried out in isopropanol (IPA) and water.

In some embodiments, the volume ratio of IPA and water is about 1:1 to about 10:1. In some embodiments, the volume ratio of IPA and water is about 1:1 to about 5:1. In some embodiments, the volume ratio of IPA and water is about 1: 1 to about 3:1. In some embodiments, the volume ratio of IPA and water is about 2: 1 to about 3:1. In some embodiments, the volume ratio of IPA and water is about 2: 1 to about 2.6:1. In some embodiments, the volume ratio of IPA and water is about 2.1: 1 to about 2.5:1. In some embodiments, the volume ratio of IPA and water is about 2.2:1 to about 2.4: 1. In some embodiments, the volume ratio of IPA and water is about 2.25:1 to about 2.35:1. In some embodiments, the volume ratio of IPA and water is about 2.3:1.

In some embodiments, cyclizing the compound of Formula F2 with a compound of Formula F3 in the presence of sodium iodide in Step b) is carried out at an elevated temperature (i.e., above ambient temperature). In some embodiments, cyclizing the compound of Formula F2 with a compound of Formula F3 is carried out at a temperature ranging from about 20° C. to about 60° C. In some embodiments, cyclizing the compound of Formula F2 with a compound of

Formula F3 is carried out at a temperature ranging from about 25° C. to about 50° C. In some embodiments, cyclizing the compound of Formula F2 with a compound of Formula F3 is carried out at a temperature ranging from about 30° C. to about 45° C. In some embodiments, cyclizing the compound of Formula F2 with a compound of Formula F3 is carried out a temperature ranging from about 35° C. to about 45° C. In some embodiments, cyclizing the compound of Formula F2 with a compound of Formula F3 is carried out at a temperature ranging from about 36° C. to about 48° C. In some embodiments, cyclizing the compound of Formula F2 with a compound of Formula F3 is carried out at a temperature ranging from about 39° C. to about 45° C. In some embodiments, cyclizing the compound of Formula F2 with a compound of Formula F3 is carried out at a temperature ranging from about 41° C. to about 43° C. In some embodiments, cyclizing the compound of Formula F2 with a compound of Formula F3 is carried out at a temperature of about 42° C.

In some embodiments, cyclizing the compound of Formula F2 with a compound of Formula F3 is carried out for no less than about 24 hours. In some embodiments, cyclizing the compound of Formula F2 with a compound of Formula F3 is carried out for about 24 hours.

### Step c)—Processes for Preparing 3-isobutyl-9,10-dimethoxy-2,3,4,6,7,11b-hexahydro-1H-pyrido[2,1-a]isoquinolin-2-ol (Compound of Formula F5)

In some embodiments, the compound of Formula F5 is prepared by the processes described herein comprising reducing the compound of Formula F4 (for example, prepared as described in Step b)) with a reducing agent to afford the compound of Formula F5:

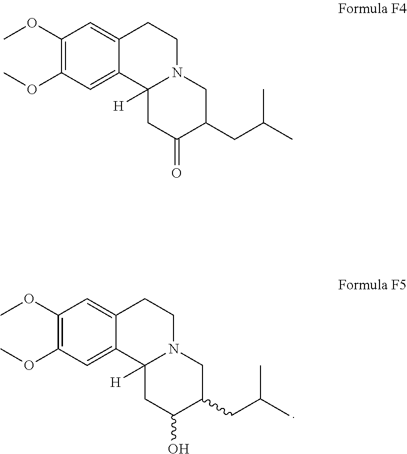

In some embodiments, reducing the compound of Formula F4 with a reducing agent in Step c) is carried out in a reducing-step solvent. The reducing-step solvent can be any suitable solvent. In some embodiments, reducing the compound of Formula F4 with a reducing agent in Step c) is carried out in a reducing-step solvent comprising methyl tert-butyl ether (MTBE) and methanol. In some embodiments, reducing the compound of Formula F4 with a reducing agent in Step c) is carried out in methyl tert-butyl ether (MTBE) and methanol.

In some embodiments, the volume ratio of MTBE and methanol is from about 1:1 to about 10:1. In some embodiments, the volume ratio of MTBE and methanol is from about 1:1 to about 5:1. In some embodiments, the volume ratio of MTBE and methanol is from about 3:1 to about 7:1. In some embodiments, the volume ratio of MTBE and methanol is from about 3:1 to about 5:1. In some embodiments, the volume ratio of MTBE and methanol is about 4.4:1.

In some embodiments, reducing the compound of Formula F4 with a reducing agent in Step c) is carried out in the presence of an organic acid. In some embodiments, the acid is acetic acid, formic acid, oxalic acid, maleic acid, lactic acid, ascorbic acid, mandelic acid, or a mixture thereof. In some embodiments, the organic acid is acetic acid.

In some embodiments, the solvent comprising methyl tert-butyl ether (MTBE) and methanol further comprises an acid. In some embodiments, the acid comprises acetic acid. In some embodiments, the acid is acetic acid.

In some embodiments, the acetic acid is present in excess (on a molar basis) compared to the compound of Formula F4.

In some embodiments, reducing the compound of Formula F4 with a reducing agent in Step c) is carried out in methyl tert-butyl ether (MTBE), acetic acid, and methanol.

In some embodiments, the molar ratio of acetic acid to the compound of Formula F4 is about 0.5 to about 1.5. In some embodiments, the molar ratio of acetic acid to the compound of Formula F4 is about 0.8 to about 1.3. In some embodiments, the molar ratio of acetic acid to the compound of Formula F4 is about 0.9 to about 1.2. In some embodiments, the molar ratio of acetic acid to the compound of Formula F4 is about 1.0 to about 1.2. In some embodiments, the molar ratio of acetic acid to the compound of Formula F4 is about 1.1.

In some embodiments, the reducing agent is added as a slurry in MTBE to the compound of Formula F4. In some embodiments, the reducing agent is added to the compound of Formula F4 as a solid. In some embodiments, the reducing agent is a borohydride reducing agent. In some embodiments, the reducing agent is a borohydride. In some embodiments, the reducing agent is sodium borohydride, lithium borohydride, calcium borohydride, magnesium borohydride, potassium borohydride, 9-BBN, cyano borohydride, bis-triphenylphosphine borohydride, sodium triethyl borohydride, tetrabutylammonium borohydride, tetramethylammonium borohydride, tetraethylammonium borohydride, or lithium tricthyl borohydride.

In some embodiments, the reducing agent in Step c) is sodium borohydride.

In some embodiments, the molar ratio of sodium borohydride to the compound of Formula F4 is about 1.0 to about 10.0. In some embodiments, the molar ratio of sodium borohydride to the compound of Formula F4 is about 1.0 to about 5.0. In some embodiments, the molar ratio of sodium borohydride to the compound of Formula F4 is about 1.0 to about 3.0. In some embodiments, the molar ratio of sodium borohydride to the compound of Formula F4 is about 1.5 to about 2.5. In some embodiments, the molar ratio of sodium borohydride to the compound of Formula F4 is about 1.8 to about 2.2. In some embodiments, the molar ratio of sodium borohydride to the compound of Formula F4 is about 1.9 to about 2.1. In some embodiments, the molar ratio of sodium borohydride to the compound of Formula F4 is about 2.0.

In some embodiments, reducing the compound of Formula F4 with a reducing agent in Step c) is conducted at a temperature during the addition of the reducing agent from about minus 5° C. to about minus 15° C., from about minus 5° C. to about minus 10° C., from about minus 5° C. to about 0° C., from about 0° C. to about 5° C., from about 0 to about 10° C., from about 0° C. to about 15° C., from about 0° C. to about 25° C., from about 0° C. to about 30° C., from about 5° C. to about 30° C., from about 10° C. to about 30° C., from about 20° C. to about 30° C., from about 20° C. to about 25° C., from about 20° C. to about 24° C., and from about 21° C. to about 23° C.

In some embodiments, reducing the compound of Formula F4 with a reducing agent in Step c) is conducted at a temperature of about 25° C. after the addition of the reducing agent. In some embodiments, reducing the compound of Formula F4 in Step c) is conducted over a period of about 2 hours after the addition of the reducing agent. In some embodiments, reducing the compound of Formula F4 in Step c) is conducted at a temperature ranging from about 15° C. to about 30° C. and over a period of at least 1.5 hours after the addition of the reducing agent. In some embodiments, reducing the compound of Formula F4 in Step c) is conducted at a temperature ranging from about 15° C. to about 30° C. and over a period of about 1 hours to about 3 hours after the addition of the reducing agent. In some embodiments, reducing the compound of Formula F4 in Step c) is conducted at a temperature ranging from about 18° C. to about 28° C. and over a period of about 1.5 hours to about 2.5 hours after the addition of the reducing agent. In some embodiments, reducing the compound of Formula F4 in Step c) is conducted at a temperature ranging from about 20° C. to about 28° C. and over a period of about 1.8 hours to about 2.2 hours after the addition of the reducing agent.

In some embodiments, lithium chloride is not present in the reacting of a compound of Formula F4 with a reducing agent in Step c).

### Step d)—Processes for preparing (2R,3R,11bR)-3-isobutyl-9,10-dimethoxy-2,3,4,6,7,11b-hexahydro-1H-pyrido[2,1-a]isoquinolin-2-ol (S)-(+)-camphorsulfonate (compound of Formula F6-CSA)

In some embodiments, the compound of Formula F6-CSA is prepared by the processes described herein comprising resolving the compound of Formula F5 (for example, prepared as described in Step c)) with (S)-(+)-camphorsulfonic acid (CSA) to afford the compound of Formula F6-CSA:

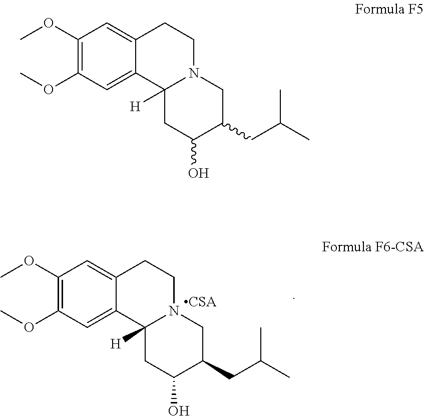

In some embodiments, the molar ratio of CSA to the compound of Formula F5 is about 0.6:1 to about 1:1. In some embodiments, the molar ratio of CSA to the compound of Formula F5 is about 0.66:1 to about 0.99:1. In some embodiments, the molar ratio of CSA to the compound of Formula F5 is about 0.70: 1 to about 0.95:1. In some embodiments, the molar ratio of CSA to the compound of Formula F5 is about 0.74: 1 to about 0.91:1. In some embodiments, the molar ratio of CSA to the compound of Formula F5 is about 0.76:1 to about 0.89:1. In some embodiments, the molar ratio of CSA to the compound of Formula F5 is about 0.78:1 to about 0.87:1. In some embodiments, the molar ratio of CSA to the compound of Formula F5 is about 0.80: 1 to about 0.85:1. In some embodiments, the molar ratio of CSA to the compound of Formula F5 is about 0.81:1 to about 0.84:1.

In some embodiments, resolving the compound of Formula F5 with (S)-(+)-camphorsulfonic acid (CSA) in Step d) is carried out in a resolving-step solvent. The resolving-step solvent can be any suitable solvent. In some embodiments, resolving the compound of Formula F5 with (S)-(+)-camphorsulfonic acid (CSA) in Step d) is carried out in a resolving-step solvent comprising an alcohol and water. In some embodiments, resolving the compound of Formula F5 with (S)-(+)-camphorsulfonic acid (CSA) in Step d) is carried out in a resolving-step solvent comprising ethanol and water. In some embodiments, resolving the compound of Formula F5 with (S)-(+)-camphorsulfonic acid (CSA) in Step d) is carried out in ethanol and water.

In some embodiments, the resolving-step solvent comprises water and ethanol in a volume ratio of water to ethanol of about 1:5 to about 1:25. In some embodiments, the resolving-step solvent comprises water and ethanol in a volume ratio of water to ethanol of about 1:10 to about 1:20. In some embodiments, the resolving-step solvent comprises water and ethanol in a volume ratio of water to ethanol of about 1:14 to about 1:18. In some embodiments, the resolving-step solvent comprises water and ethanol in a volume ratio of water to ethanol of about 1:15 to about 1:17. In some embodiments, the resolving-step solvent comprises water and ethanol in a volume ratio of water to ethanol of about 1:15.5 to about 1:16.5. In some embodiments, the resolving-step solvent comprises water and ethanol in a volume ratio of water to ethanol of about 1:16.

In some embodiments, the resolving-step solvent is about 10 to about 14 volumes of ethanol and about 0.5 to about 1.0 volumes of water. In some embodiments, the resolving-step solvent is about 11 to about 13 volumes of ethanol and about 0.65 to about 0.85 volumes of water. In some embodiments, the resolving-step solvent comprises about 12 volumes of ethanol and about 0.75 volumes of water.

In some embodiments, resolving the compound of Formula F5 with (S)-(+)-camphorsulfonic acid (CSA) in Step d) is conducted at a temperature ranging from about 55° C. to about 78° C., about 60° C. to about 75° C., about 65° C. to about 73° C., about 67° C. to about 72° C., or about 69° C. to about 71° C. In some embodiments, resolving the compound of Formula F5 takes place at a temperature of about 70° C.

In some embodiments, resolving the compound of Formula F5 further comprises 1) heating to a first temperature in the presence of CSA, and 2) cooling to a second temperature. In some embodiments, the first temperature is at a temperature ranging from about 55° C. to about 78 ° ° C., about 60° C. to about 75° C., about 65° C. to about 73 ºC, about 67° C. to about 72° C., or about 69° C. to about 71° C. In some embodiments, the second temperature is at a temperature ranging from about 10° C. to about 32° C., about 12° C. to about 30° C., about 15° C. to about 28° C., about 18° C. to about 26° C., or about 20° C. to about 24° C. In some embodiments, the cooling step is conducted at a rate ranging from about 2° C./hr. to about 4° C./hr. In some embodiments, the cooling step is conducted at a rate of about 3° C./hr.

In some embodiments, the reaction mixture of Formula F5 and CSA is cooled to about 22 ° C. In some embodiments, the reaction mixture is seeded with a crystal of the compound of Formula F6-CSA. In some embodiments, the compound of Formula F6-CSA is dried under vacuum at an elevated temperature (i.e., above 25° C.). In some embodiments, the compound of Formula F6-CSA is dried under vacuum at about 45° C. for no less than 12 hours.

In some embodiments, the compound of Formula F6-CSA prepared from Step d) has an optical purity of about 95% or greater, about 96% or greater, about 97% or greater, about 97.5% or greater, about 98% or greater, about 98.5% or greater, about 99% or greater, about 99.1% or greater, about 99.2% or greater, about 99.3% or greater, about 99.4% or greater, about 99.5% or greater, about 99.6% or greater, about 99.7% or greater, about 99.8% or greater, or about 99.9% or greater. In some embodiments, the compound of Formula F6-CSA has an optical purity of about 99% or greater.

### Step e)—Processes for Preparing (2R,3R,11bR)-3-isobutyl-9,10-dimethoxy-2,3,4,6,7,11b-hexahydro-1H-pyrido[2,1-a]isoquinolin-2-ol (Compound of Formula F6, Free Base)

In some embodiments, the compound of Formula F6 is prepared by the processes described herein comprising reacting the compound of Formula F6-CSA (for example, prepared as described in Step d)) with a Step e)-base to afford the compound of Formula F6:

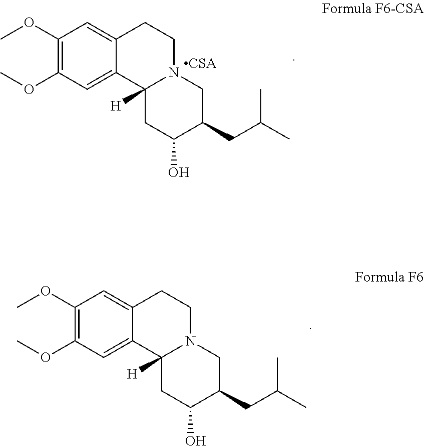

In some embodiments, the Step e)-base is an inorganic base. In some embodiments, the Step e)-base is sodium bicarbonate, sodium carbonate, sodium citrate, sodium hydroxide, or potassium hydroxide. In some embodiments, the Step e)-base is potassium hydroxide. In some embodiments, the Step e)-base is aqueous potassium hydroxide. In some embodiments, the Step e)-base is 2N aqueous potassium hydroxide. In some embodiments, the Step e)-base is sodium hydroxide. In some embodiments, the Step e)-base is aqueous sodium hydroxide. In some embodiments, the Step e)-base is 1N aqueous sodium hydroxide.

In some embodiments, reacting the compound of Formula F6-CSA with a Step e)-base is carried out in the presence of a Step e)-solvent. The Step e)-solvent can be any suitable solvent. In some embodiments, the Step e)-solvent is a solvent comprising a hydrocarbon, chlorinated hydrocarbon, alcohol, ether, ester, carbonate, amide, nitrile, sulfoxide, sulfone, nitro compound, heteroarene, heterocycle, water, or a mixture thereof. In some embodiments, the Step e)-solvent is a chlorinated hydrocarbon solvent. In some embodiments, the Step e)-solvent is an ether. In some embodiments, the Step e)-solvent is a cycloalkyl ether. In some embodiments, the Step e)-solvent is 2-methyltetrahydrofuran (MeTHF). In some embodiments, the Step e)-solvent comprises water and a halogenated hydrocarbon solvent. In some embodiments, the halogenated hydrocarbon solvent is dichloromethane.

In some embodiments, reacting the compound of Formula F6-CSA with a Step e)-base is carried out in the presence of a Step e)-solvent comprising 2-methyltetrahydrofuran (MeTHF). In some embodiments, reacting the compound of Formula F6-CSA with a Step e)-base is carried out in the presence of 2-methyltetrahydrofuran (MeTHF). In some embodiments, reacting the compound of Formula F6-CSA with a Step e)-base is carried out in the presence of 2-methyltetrahydrofuran (MeTHF), wherein the Step e)-base is potassium hydroxide. In some embodiments, reacting the compound of Formula F6-CSA with a Step e)-base is carried out in the presence of 2-methyltetrahydrofuran (MeTHF), wherein the Step e)-base is aqueous potassium hydroxide. In some embodiments, reacting the compound of Formula F6-CSA with a Step e)-base is carried out in the presence of 2-methyltetrahydrofuran (MeTHF), wherein the Step e)-base is 2N aqueous potassium hydroxide.

In some embodiments, reacting the compound of Formula F6-CSA with a Step e)-base is carried out in the presence of 2-methyltetrahydrofuran (MeTHF) and water.

In some embodiments, reacting the compound of Formula F6-CSA with a Step e)-base is carried out in the presence of a Step e)-solvent comprising dichloromethane. In some embodiments, reacting the compound of Formula F6-CSA with a Step e)-base is carried out in the presence of dichloromethane. In some embodiments, reacting the compound of Formula F6-CSA with a Step e)-base is carried out in the presence of dichloromethane, wherein the Step e)-base is sodium hydroxide. In some embodiments, reacting the compound of Formula F6-CSA with a Step c)-base is carried out in the presence of dichloromethane, wherein the Step e)-base is aqueous sodium hydroxide. In some embodiments, reacting the compound of Formula F6-CSA with a Step e)-base is carried out in the presence of dichloromethane, wherein the Step e)-base is 1N aqueous sodium hydroxide. In some embodiments, the Step e)-base is 1N sodium hydroxide.

In some embodiments, reacting the compound of Formula F6-CSA with a Step e)-base is carried out in the presence of a Step c)-solvent comprising dichloromethane and water.

In some embodiments, reacting the compound of Formula F6-CSA with a Step e)-base is carried out in the presence of dichloromethane. In some embodiments, reacting the compound of Formula F6-CSA with a Step e)-base is carried out in the presence of dichloromethane and water.

In some embodiments, reacting the compound of Formula F6-CSA with a Step e)-base is conducted at a temperature ranging from about 20° C. to about 30° C. about 21° C. to about 29° C. about 22° C. to about 28° C. about 23° C. to about 27° C. about 24° C. to about 26° C. or about 25° C.

In some embodiments, the compound of Formula F6 is not isolated. Accordingly, in some embodiments, reacting the compound of Formula F6-CSA with a Step e)-base is conducted in the presence of a Step e)-solvent, and after completion of the reaction, the mixture of the compound of Formula F6 and the Step e)-solvent is used directly in Step f). In some embodiments, the Step c)-solvent is dichloromethane. In some embodiments, the Step e)-solvent is 2-methyltetrahydrofuran (MeTHF).

### Step f)—Processes for Preparing (S)-(2R,3R,11bR)-3-isobutyl-9,10-dimethoxy-2,3,4,6,7,11b-hexahydro-1/-pyrido[2,1-a]isoquinolin-2-yl 2-((tert-butoxycarbonyl)amino)-3-methylbutanoate (Compound of Formula F8)

In some embodiments, the compound of Formula F8 is prepared by the processes described herein comprising coupling the compound of Formula F6 (for example, prepared as described in Step e)) and a carboxylic acid of Formula F7 with a coupling reagent to afford the compound of Formula F8:

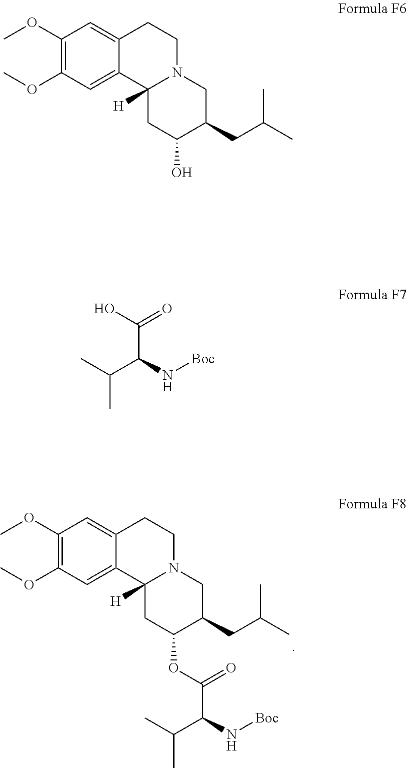

In some embodiments, coupling the compound of Formula F6 and a carboxylic acid of Formula F7 with a coupling reagent in Step f) is carried out in the presence of a coupling-step base. In some embodiments, the coupling-step base is an organic base.

In some embodiments, coupling the compound of Formula F6 and a carboxylic acid of Formula F7 with a coupling reagent in Step f) is carried out in the presence of a coupling-step base comprising 4-dimethylaminopyridine (DMAP). In some embodiments, coupling the compound of Formula F6 and a carboxylic acid of Formula F7 with a coupling reagent in Step f) is carried out in the presence of 4-dimethylaminopyridine (DMAP).

In some embodiments, the coupling-step base is present in a catalytic amount (i.e., less than the molar quantity of the compound of Formula F6). In some embodiments, the molar ratio of the coupling-step base to the compound of Formula F6 is about 0.6:1.0, about 0.51.0, about 0.4:1.0, about 0.3:1.0, about 0.27: 1.0, or about 0.25:1.0. In some embodiments, the molar ratio of 4-dimethylaminopyridine (DMAP) to the compound of Formula F6 is about 0.6:1.0, about 0.5:1.0, about 0.4:1.0, about 0.3:1.0, about 0.27:1.0, or about 0.25:1.0.

In some embodiments, coupling the compound of Formula F6 and a carboxylic acid of Formula F7 with a coupling reagent in Step f) is carried out in the presence of a coupling-step solvent. The coupling-step solvent can be any suitable solvent. In some embodiments, the coupling-step solvent is a hydrocarbon, chlorinated hydrocarbon, alcohol, ether, ester, carbonate, amide, nitrile, sulfoxide, sulfone, nitro compound, heteroarene, heterocycle, water, or a mixture thereof. In some embodiments, the solvent is a chlorinated hydrocarbon solvent. In some embodiments, the solvent is dichloromethane. In some embodiments, the solvent is an ether. In some embodiments, the solvent is a cycloalkyl ether. In some embodiments, the solvent is 2-methyltetrahydrofuran (MeTHF).

In some embodiments, coupling the compound of Formula F6 and a carboxylic acid of Formula F7 with a coupling reagent in Step f) is carried out in the presence of a coupling-step solvent comprising dichloromethane. In some embodiments, coupling the compound of Formula F6 and a carboxylic acid of Formula F7 with a coupling reagent in Step f) is carried out in the presence of dichloromethane. In some embodiments, coupling the compound of Formula F6 and a carboxylic acid of Formula F7 with a coupling reagent in Step f) is carried out in the presence of a coupling-step solvent comprising 2-methyltetrahydrofuran (MeTHF). In some embodiments, coupling the compound of Formula F6 and a carboxylic acid of Formula F7 with a coupling reagent in Step f) is carried out in the presence of 2-methyltetrahydrofuran (MeTHF).

In some embodiments, the coupling reagent is a carbodiimide, 1,1′-carbonyldiimidazole (CDI), bis(2-oxo-3-oxazolidinyl)phosphinic chloride (BOP-Cl), hexafluorophosphate (BOP reagent), PCh, PCls, or 1-propanephosphonic acid cyclic anhydride. In some embodiments, the coupling reagent is N-(3-dimethylaminopropyl)-N′-ethylcarbodiimide (EDC or EDCI), N-(3-dimethylaminopropyl)-N′-ethylcarbodiimide hydrochloride (EDC hydrochloride), 1-[3-(dimethylamino)propyl]-3-ethylcarbodiimide methiodide (EDC methiodide), 1-cyclohexyl-3-(2-morpholinoethyl)carbodiimide metho-p-toluenesulfonate, or 1,3-dicyclohexylcarbodiimide (DCC). In some embodiments, the coupling reagent is N-(3-dimethylaminopropyl)-N-ethylcarbodiimide (EDC or EDCI), N-(3- dimethylaminopropyl)-N-ethylcarbodiimide hydrochloride (EDC hydrochloride), 1-[3-(dimethylamino)propyl]-3-ethylcarbodiimide methiodide (EDC methiodide), 1-cyclohexyl-3-(2-morpholinoethyl)carbodiimide metho-p-toluenesulfonate, or 1,3-dicyclohexykarbodiimide (DCC).

In some embodiments, the coupling reagent is N-(3-dimethylaminopropyl)-N′-ethylcarbodiimide (EDC or EDCI). In some embodiments, the coupling reagent is N-(3-dimethylaminopropyl)-N′-ethylcarbodiimide hydrochloride (EDC. HCl).

In some embodiments, coupling the compound of Formula F6 and a carboxylic acid of Formula F7 is conducted at a temperature below about 25° C. In some embodiments, coupling the compound of Formula F6 and a carboxylic acid of Formula F7 is conducted at a temperature ranging from about −10° C. to about 30° C., from about −10° C. to about 25° C., about −5° C. to about 20° C., about −5° C. to about 15° C., about −5° C. to about 10° C., or about −1° C. to about 25° C.

### Step g)—Processes for Preparing (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate dihydrochloride (Compound of Formula F9-HCl).

In some embodiments, the compound of Formula F9-HCl is prepared by the processes described herein comprising deprotecting the compound of Formula F8 (for example, prepared as described in Step f)) with hydrogen chloride to afford the compound of Formula F9-HCl:

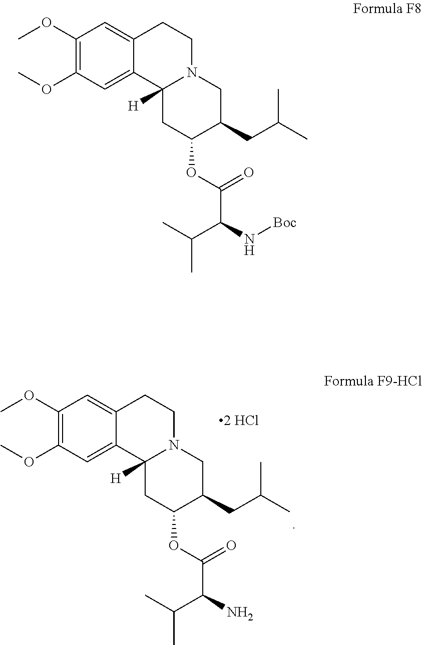

In some embodiments, the hydrogen chloride in Step g) is hydrogen chloride gas. In some embodiments, the hydrogen chloride in Step g) is aqueous hydrogen chloride (i.e., hydrochloric acid). In some embodiments, the hydrogen chloride in Step g) is a mixture of hydrogen chloride and any suitable organic solvent. In some embodiments, the hydrogen chloride in Step g) comprises a hydrogen chloride isopropanol (IPA) mixture. In some embodiments, the hydrogen chloride in Step g) is a hydrogen chloride isopropanol (IPA) mixture. In some embodiments, the hydrogen chloride in Step g) is a 3.7M hydrogen chloride isopropanol (IPA) mixture. In some embodiments, the hydrogen chloride in Step g) is a 3.7M solution of hydrogen chloride in isopropanol (IPA). In some embodiments, the hydrogen chloride in Step g) comprises a hydrogen chloride dioxane mixture. In some embodiments, the hydrogen chloride in Step g) is a hydrogen chloride dioxane mixture. In some embodiments, the hydrogen chloride in Step g) is a 4M hydrogen chloride dioxane mixture. In some embodiments, the hydrogen chloride in Step g) is a 4M solution of hydrogen chloride in dioxane. In some embodiments, the hydrogen chloride in Step g) is substantially anhydrous.

In some embodiments, deprotecting the compound of Formula F8 with hydrogen chloride in Step g) is carried out in a deprotecting-step solvent. A deprotecting-step solvent can be any suitable solvent. In some embodiments, deprotecting the compound of Formula F8 with hydrogen chloride in Step g) is carried out in a deprotecting-step solvent comprising dichloromethane. In some embodiments, deprotecting the compound of Formula F8 with hydrogen chloride in Step g) is carried out in a deprotecting-step solvent comprising 2-methyltetrahydrofuran (MeTHF). In some embodiments, deprotecting the compound of Formula F8 with hydrogen chloride in Step g) is carried out in a deprotecting-step solvent comprising ethyl acetate (EtOAc). In some embodiments, deprotecting the compound of Formula F8 with hydrogen chloride in Step g) is carried out in a deprotecting-step solvent comprising 2-methyltetrahydrofuran (MeTHF) and ethyl acetate (EtOAc). In some embodiments, deprotecting the compound of Formula F8 with hydrogen chloride in Step g) is carried out in dichloromethane. In some embodiments, deprotecting the compound of Formula F8 with hydrogen chloride in Step g) is carried out in methyl tert-butyl ether (MTBE). In some embodiments, deprotecting the compound of Formula F8 with hydrogen chloride in Step g) is carried out in methyl tert-butyl ether (MTBE) and ethyl acetate (EtOAc). In some embodiments, deprotecting the compound of Formula F8 with hydrogen chloride in Step g) is carried out in dichloromethane and dioxane. In some embodiments, deprotecting the compound of Formula F8 with hydrogen chloride in Step g) is carried out in dichloromethane and isopropanol. In some embodiments, deprotecting the compound of Formula F8 with hydrogen chloride in Step g) is carried out in 2-methyltetrahydrofuran (MeTHF) and dioxane. In some embodiments, deprotecting the compound of Formula F8 with hydrogen chloride in Step g) is carried out in 2-methyltetrahydrofuran (MeTHF) and isopropanol. In some embodiments, deprotecting the compound of Formula F8 with hydrogen chloride in Step g) is carried out in 2-methyltetrahydrofuran (MeTHF), ethyl acetate (EtOAc), and dioxane. In some embodiments, deprotecting the compound of Formula F8 with hydrogen chloride in Step g) is carried out in 2-methyltetrahydrofuran (MeTHF), ethyl acetate (EtOAc), and isopropanol.

In some embodiments, Step g) further comprises a “solvent swap” where the solvent used in the deprotection of the compound of Formula F8 is different from the solvent that affords the isolated compound of Formula F9-HCl. It is understood that after the deprotection with hydrogen chloride the compound of Formula F9-HCl is initially formed and can either be isolated directly or subsequently neutralized to form the free base (the compound of Formula F9) prior to the “solvent swap”. After the solvent swap, the free base can be converted to the compound of Formula F9-HCl with hydrogen chloride. Accordingly, in some embodiments, after deprotecting the compound of Formula F8 with hydrogen chloride, Step g) further comprises the steps:
1) reacting the compound of Formula F9-HCl with a base to afford a compound of Formula F9 (free base):

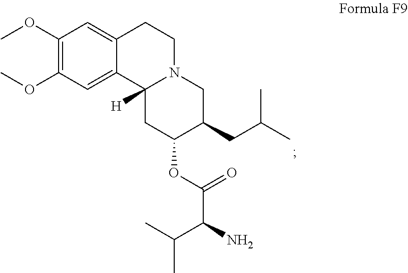 [SMILES: COc1cc2c(cc1OC)[C@H]1C[C@@H](OC(=O)[C@@H](N)C(C)C)[C@H](CC(C)C)CN1CC2]

and
2) reacting the compound of Formula F9 (free base) with hydrogen chloride to afford the compound of Formula F9-HCl.

In some embodiments, with reference to Step g)-Step 1), the base is an inorganic base. In some embodiments, the base is sodium bicarbonate, sodium carbonate, sodium citrate, sodium hydroxide, or potassium hydroxide. In some embodiments, the base is potassium hydroxide. In some embodiments, the base is sodium hydroxide. In some embodiments, the base is sodium bicarbonate. In some embodiments, the base is aqueous sodium bicarbonate. In some embodiments, with reference to Step g)-Step 1), reacting the compound of Formula F9-HCl with a base to afford a compound of Formula F9 (free base) is conducted in the presence of a solvent. In some embodiments, the solvent comprises dichloromethane. In some embodiments, the solvent comprises dichloromethane and dioxane. In some embodiments, the solvent is a mixture of dichloromethane and dioxane. In some embodiments, the compound of Formula F9 (free base) is isolated as a mixture comprising dichloromethane.

In some embodiments, with reference to Step g)-Step 2), reacting the compound of Formula F9 with hydrogen chloride to afford the compound of Formula F9-HCl is conducted in the presence of a solvent. In some embodiments, the solvent comprises acetonitrile. In some embodiments, the solvent comprises acetonitrile and isopropanol. In some embodiments, the solvent is a mixture of acetonitrile, isopropanol, and ethyl acetate. In some embodiments, the hydrogen chloride is a hydrogen chloride isopropanol mixture. In some embodiments, the hydrogen chloride is a 3.7M hydrogen chloride isopropanol mixture. In some embodiments, the hydrogen chloride is a 3.7M solution of hydrogen chloride in isopropanol. In some embodiments, the hydrogen chloride is substantially anhydrous.

In some embodiments, after deprotecting the compound of Formula F8 with hydrogen chloride, Step g) further comprises the steps:
1) reacting the compound of Formula F9-HCl in a solvent comprising dichloromethane and dioxane with a base to afford a compound of Formula F9 (free base):

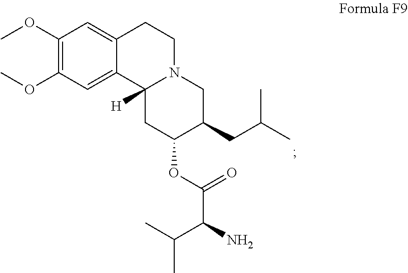 [SMILES: COc1cc2c(cc1OC)[C@H]1C[C@@H](OC(=O)[C@@H](N)C(C)C)[C@H](CC(C)C)CN1CC2]

and
2) reacting the compound of Formula F9 (free base) in a solvent comprising acetonitrile with a hydrogen chloride isopropanol mixture to afford the compound of Formula F9-HCl.

In some embodiments, after deprotecting the compound of Formula F8 with hydrogen chloride, Step g) further comprises the steps:
1) reacting the compound of Formula F9-HCl with aqueous sodium bicarbonate in the presence of a solvent comprising dichloromethane to afford a compound of Formula F9 (free base); and
2) reacting the compound of Formula F9 (free base) in a solvent comprising acetonitrile with a hydrogen chloride isopropanol mixture to afford the compound of Formula F9-HCl.

In some embodiments, after Step g) and before Step h) the process further comprises the steps of:
1) reacting the compound of Formula F9-HCl with aqueous sodium bicarbonate in the presence of a solvent comprising dichloromethane to afford a compound of Formula F9 (free base); and
2) reacting the compound of Formula F9 (free base) in a solvent comprising acetonitrile with a hydrogen chloride isopropanol mixture to afford the compound of Formula F9-HCl.

In some embodiments, the compound of Formula F9-HCl is isolated. In some embodiments, the compound of Formula F9-HCl is a solid. In some embodiments, the compound of Formula F9-HCl is crystalline. In some embodiments, the compound of Formula F9-HCl is crystalline Form I, crystalline Form II, or an amorphous solid as described in WO2017/075340, which is incorporated by reference in its entirety (for example, see Formula II (Valbenazine dihydrochloride) and Examples 14, 15, and 16 in WO2017/075340). In some embodiments, the compound of Formula F9-HCl is crystalline Form I. In some embodiments, the compound of Formula F9-HCl is crystalline Form II. In some embodiments, the compound of Formula F9-HCl is an amorphous solid.

In some embodiments, the compound of Formula F9-HCl is not isolated and used directly in Step h).

### Step h)—Processes for Preparing (S)-(2R,3R,11bR)-3-isobutyl-9,10-dimethoxy-2,3,4,6,7,11b-hexahydro-1/-pyrido[2,1-a]isoquinolin-2-yl 2-amino-3-methylbutanoate (Compound of

Formula F9, Free Base)

In some embodiments, the compound of Formula F9 is prepared by the processes described herein comprising reacting the compound of Formula F9-HCl (for example, prepared as described in Step g)) with a Step h)-base to afford a compound of Formula F9 (free base):

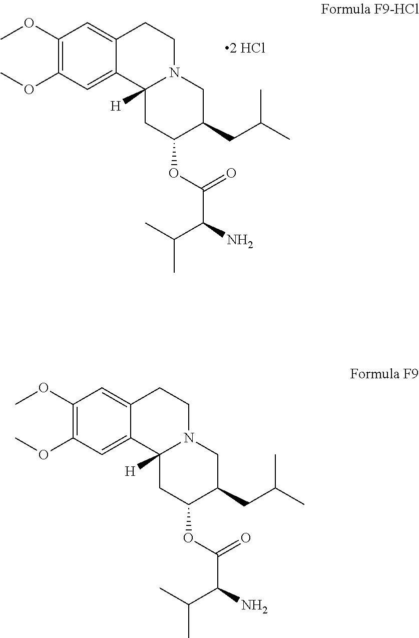

In some embodiments, the Step h)-base is an inorganic base. In some embodiments, the Step h)-base is sodium bicarbonate, sodium carbonate, sodium citrate, sodium hydroxide, or potassium hydroxide. In some embodiments, the Step h)-base is potassium hydroxide. In some embodiments, the Step h)-base is sodium hydroxide. In some embodiments, the Step h)-base is sodium bicarbonate. In some embodiments, the Step h)-base is aqueous sodium bicarbonate.

In some embodiments, reacting the compound of Formula F9-HCl with a Step h)-base is carried out in the presence of a Step h)-solvent. The Step h)-solvent can be any suitable solvent. In some embodiments, the Step h)-solvent is a solvent comprising a hydrocarbon, chlorinated hydrocarbon, alcohol, ether, ester, carbonate, amide, nitrile, sulfoxide, sulfone, nitro compound, heteroarene, heterocycle, water, or a mixture thereof In some embodiments, the Step h)-solvent is an ether. In some embodiments, the Step h)-solvent is a cycloalkyl ether. In some embodiments, the Step h)-solvent is 2-methyltetrahydrofuran (MeTHF). In some embodiments, the Step h)-solvent comprises water and a halogenated hydrocarbon solvent. In some embodiments, the halogenated hydrocarbon solvent is dichloromethane.

In some embodiments, reacting the compound of Formula F9-HCl with a Step h)-base is carried out in the presence of a Step h)-solvent comprising dichloromethane. In some embodiments, reacting the compound of Formula F9-HCl with a Step h)-base is carried out in the presence of dichloromethane. In some embodiments, reacting the compound of Formula F9-HCl with a Step h)-base is carried out in the presence of dichloromethane and water. In some embodiments, reacting the compound of Formula F9-HCl with a Step h)-base is carried out in the presence of dichloromethane, wherein the Step h)-base is aqueous sodium bicarbonate.

In some embodiments, reacting the compound of Formula F9-HCl with a Step h)-base is carried out in the presence of a Step h)-solvent comprising 2-methyltetrahydrofuran (MeTHF). In some embodiments, reacting the compound of Formula F9-HCl with a Step h)-base is carried out in the presence of a Step h)-solvent comprising ethyl acetate (EtOAc). In some embodiments, reacting the compound of Formula F9-HCl with a Step h)-base is carried out in the presence of a Step h)-solvent comprising 2-methyltetrahydrofuran (MeTHF) and ethyl acetate (EtOAc). In some embodiments, reacting the compound of Formula F9-HCl with a Step h)-base is carried out in 2-methyltetrahydrofuran (MeTHF). In some embodiments, reacting the compound of Formula F9-HCl with a Step h)-base is carried out in ethyl acetate (EtOAc). In some embodiments, reacting the compound of Formula F9-HCl with a Step h)-base is carried out in 2-methyltetrahydrofuran (MeTHF) and ethyl acetate (EtOAc).

In some embodiments, reacting the compound of Formula F9-HCl with a Step h)-base is carried out in 2-methyltetrahydrofuran (MeTHF) and water. In some embodiments, reacting the compound of Formula F9-HCl with a Step h)-base is carried out in ethyl acetate (EtOAc) and water. In some embodiments, reacting the compound of Formula F9-HCl with a Step h)-base is carried out in 2-methyltetrahydrofuran (MeTHF), ethyl acetate (EtOAc), and water.

In some embodiments, reacting the compound of Formula F9-HCl with a Step h)-base is carried out in 2-methyltetrahydrofuran (MeTHF), wherein the Step h)-base is aqueous sodium bicarbonate. In some embodiments, reacting the compound of Formula F9-HCl with a Step h)-base is carried out in ethyl acetate (EtOAc), wherein the Step h)-base is aqueous sodium bicarbonate. In some embodiments, reacting the compound of Formula F9-HCl with a Step h)-base is carried out in 2-methyltetrahydrofuran (MeTHF) and ethyl acetate (EtOAc), wherein the Step h)-base is aqueous sodium bicarbonate.

In some embodiments, reacting the compound of Formula F9-HCl with a Step h)-base is conducted at a temperature ranging from about 20° C. to about 30° C., about 21° C. to about 29° C., about 22° C. to about 28° C., about 23° C. to about 27° C., about 24° C. to about 26° C., or about 25° C.

In some embodiments, the compound of Formula F9 is not isolated. Accordingly, in some embodiments, reacting the compound of Formula F9-HCl with a Step h)-base is conducted in the presence of a Step h)-solvent, and after completion of the reaction, the mixture of the compound of Formula F9 and the Step h)-solvent is used directly in Step i). In some embodiments, the Step h)-solvent is dichloromethane.

### Step i)—Processes for Preparing (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Compound of Formula I)

The compound of Formula I can be prepared by any of the processes as described herein, such as, reacting p-toluenesulfonic acid with either the compound of Formula F9 (free base) or the compound of Formula F9-HCl to afford the compound of Formula I, as described herein (e.g., Step i-a) and Step i-b), respectively).

Step i-a), Utilizing the Compound of Formula F9 (Free Base).

In some embodiments, the compound of Formula I is prepared by the processes described herein comprising reacting the compound of Formula F9 (for example, prepared as described in Step h)) with p-toluenesulfonic acid to afford the compound of Formula I:

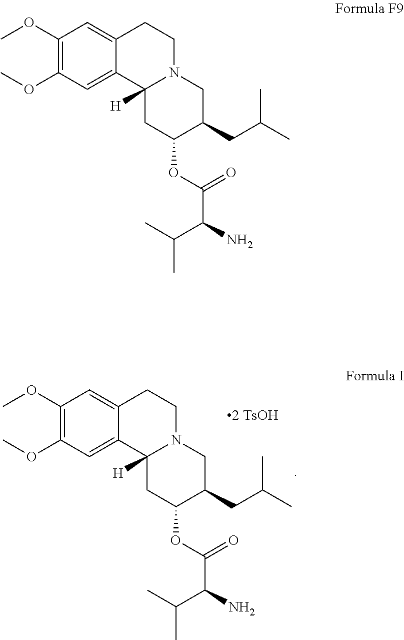

In some embodiments, reacting the compound of Formula F9 with p-toluenesulfonic acid in Step i-a) is carried out in a suitable solvent. In some embodiments, reacting the compound of Formula F9 with p-toluenesulfonic acid in Step i-a) is carried out in a solvent comprising dichloromethane.

In some embodiments, reacting the compound of Formula F9 with p-toluenesulfonic acid in Step i-a) is carried out in a solvent comprising acetonitrile. In some embodiments, the solvent comprises acetonitrile and dichloromethane. In some embodiments, reacting the compound of Formula F9 with p-toluenesulfonic acid in Step i-a) is carried out in acetonitrile.

In some embodiments, the compound of Formula F9 was not isolated and present as a mixture with the Step h)-solvent prior to reacting with p-toluenesulfonic acid. In some embodiments, the Step h)-solvent comprises dichloromethane. In some embodiments, the Step h)-solvent is dichloromethane. In some embodiments, the Step h)-solvent is “swapped” or replaced with a suitable solvent to carry out reacting the compound of Formula F9 with p-toluenesulfonic acid. In some embodiments, the solvent comprises acetonitrile. In some embodiments, the solvent is acetonitrile.

In some embodiments, p-toluenesulfonic acid is a solid. In some embodiments, the p-toluenesulfonic acid is a solution of p-toluenesulfonic acid in any suitable organic solvent. In some embodiments, the p-toluenesulfonic acid is a solution comprising p-toluenesulfonic acid and acetonitrile. In some embodiments, the p-toluenesulfonic acid is a solution of p-toluenesulfonic acid in acetonitrile.

In some embodiments, reacting the compound of Formula F9 with p-toluenesulfonic acid in Step i) is conducted at a temperature ranging from about 35° C. to about 65° C., about 40° C. to about 60° C., about 45° C. to about 55° C., about 47° C. to about 53° C., about 48° C. to about 52° C., or about 50° C. In some embodiments, reacting is conducted at a temperature ranging from about 48° C. to about 52° C. In some embodiments, reacting is conducted at a temperature of about 50° C.

Step i-b) Utilizing the Compound of Formula F9-HCl.

In some embodiments, the compound of Formula I is prepared by the processes described herein comprising reacting the compound of Formula F9-HCl (for example, prepared as described in Step g)) with p-toluenesulfonic acid to afford the compound of Formula I:

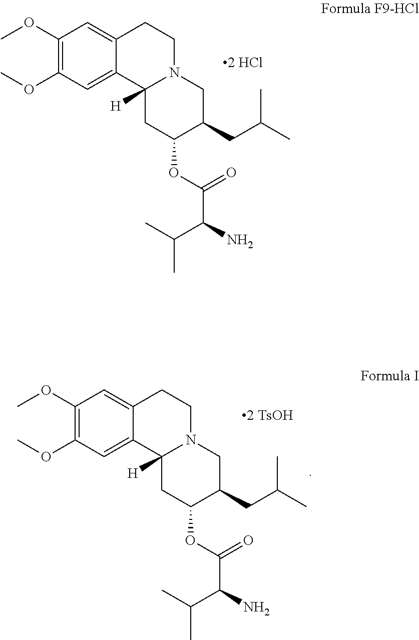

In some embodiments, the compound of Formula F9-HCl is isolated prior to use in Step i-b). In some embodiments, the compound of Formula F9-HCl prepared according to Step g) is used without isolation.

In some embodiments, reacting the compound of Formula F9-HCl with p-toluenesulfonic acid in Step i-b) is carried out in a suitable solvent. In some embodiments, reacting the compound of Formula F9-HCl with p-toluenesulfonic acid in Step i-b) is carried out in a solvent comprising ethyl acetate (EtOAc).

In some embodiments, reacting the compound of Formula F9-HCl with p-toluenesulfonic acid in Step i-b) is carried out in ethyl acetate (EtOAc). In some embodiments, the solvent is ethyl acetate and acetonitrile. In some embodiments, the solvent is ethyl acetate and dichloromethane.

In some embodiments, the compound of Formula F9-HCl was not isolated and present as a mixture with the Step g)-solvent prior to reacting with p-toluenesulfonic acid. In some embodiments, the Step g)-solvent comprises dichloromethane. In some embodiments, the Step g)-solvent is dichloromethane. In some embodiments, the Step g)-solvent is “swapped” or replaced with a suitable solvent to carry out reacting the compound of Formula F9-HCl with p-toluenesulfonic acid. In some embodiments, the solvent comprises ethyl acetate (EtOAc). In some embodiments, the solvent comprises acetonitrile. In some embodiments, the solvent is ethyl acetate (EtOAc). In some embodiments, the solvent is acetonitrile.

In some embodiments, p-toluenesulfonic acid is a solid. In some embodiments, the p-toluenesulfonic acid is a solution of p-toluenesulfonic acid in any suitable organic solvent. In some embodiments, the p-toluenesulfonic acid is a solution comprising p-toluenesulfonic acid and ethyl acetate (EtOAc). In some embodiments, the p-toluenesulfonic acid is a solution comprising p-toluenesulfonic acid and acetonitrile. In some embodiments, the p-toluenesulfonic acid is a solution of p-toluenesulfonic acid in ethyl acetate (EtOAc). In some embodiments, the p-toluenesulfonic acid is a solution of p-toluenesulfonic acid in acetonitrile.

In some embodiments, reacting the compound of Formula F9-HCl with p-toluenesulfonic acid in Step i-b) is conducted at a temperature ranging from about 25° C. to about 75° C., about 30° C. to about 75° C., about 40° C. to about 75° C., about 50° C. to about 75° C., about 60° C. to about 75° C., or about 65° C. to about 75° C., In some embodiments, reacting is conducted at a temperature of about 68° C. to about 72° C. In some embodiments, reacting is conducted at a temperature of about 70° C.

In some embodiments, the compound of Formula I is isolated. In some embodiments, the compound of Formula I is isolated by filtration. In some embodiments, the compound of Formula I is dried under vacuum at an elevated temperature. In some embodiments, the compound of Formula I is dried under vacuum at about 45° C. to about 55° C. In some embodiments, the compound of Formula I is dried under vacuum at about 45° C. to about 55° C. for no less than 12 hours. In some embodiments, the compound of Formula I is dried under vacuum at about 50° C. for no less than 12 hours. In some embodiments, the compound of Formula I is isolated and dried under vacuum at about 50° C. for no less than 12 hours.

In some embodiments the compound of Formula I has a purity of no less than about 95% by weight, no less than about 96% by weight, no less than about 97% by weight, no less than about 97.5% by weight, or no less than about 98% by weight. In some embodiments, the compound of Formula I is crystalline. In some embodiments, the compound of Formula I is crystalline Form I, crystalline Form II, crystalline Form III, crystalline Form IV, crystalline Form V, crystalline Form VI, or an amorphous solid as described in WO2017/075340 (for example, see Formula I (Valbenazine ditosylate) and Examples 2, 3, 5, 6, 7, 8, 9, 10, 11, and 16, and figures related thereto in WO2017/075340), which is incorporated by reference in its entirety. In some embodiments, the compound of Formula I is crystalline Form I. In some embodiments, the compound of Formula I is crystalline Form II. In some embodiments, the compound of Formula I is crystalline Form III. In some embodiments, the compound of Formula I is crystalline Form IV. In some embodiments, the compound of Formula I is crystalline Form V. In some embodiments, the compound of Formula I is crystalline Form VI. In some embodiments, the compound of Formula I is an amorphous solid.

Another aspect of the present invention provides processes for the preparation of a compound of Formula I:

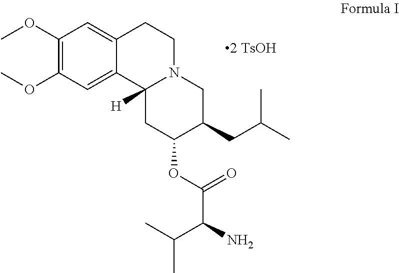 [SMILES: COc1cc2c(cc1OC)[C@H]1C[C@@H](OC(=O)[C@@H](N)C(C)C)[C@H](CC(C)C)CN1CC2]

comprising the steps of:
a) reacting a compound of Formula F1:

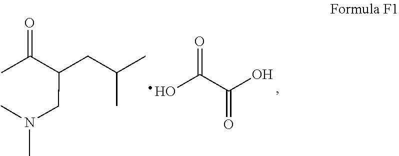

with aqueous potassium hydroxide in the presence of methyl tert-butyl ether (MTBE) to afford a compound of Formula F2:

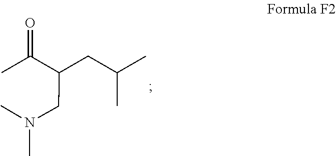

b) cyclizing the compound of Formula F2 with a compound of Formula F3:

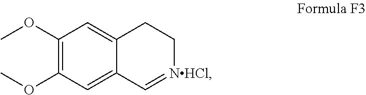

in the presence of sodium iodide, isopropanol (IPA), and water to afford a compound of Formula F4:

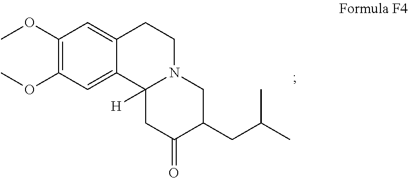 [SMILES: COc1cc2c(cc1OC)C1CC(=O)C(CC(C)C)CN1CC2]

c) reducing the compound of Formula F4 with sodium borohydride in the presence of methyl tert-butyl ether (MTBE), acetic acid, and methanol to afford a compound of Formula F5:

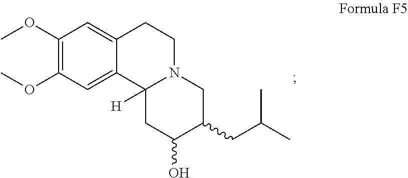 [SMILES: COc1cc2c(cc1OC)C1CC(O)C(CC(C)C)CN1CC2]

d) resolving the compound of Formula F5 with (S)-(+)-camphorsulfonic acid (CSA) in ethanol and water to afford a compound of Formula F6-CSA:

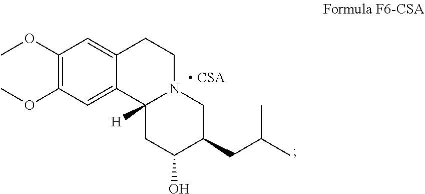 [SMILES: COc1cc2c(cc1OC)[C@H]1C[C@@H](O)[C@H](CC(C)C)CN1CC2]

e) reacting the compound of Formula F6-CSA with aqueous sodium hydroxide in dichloromethane to afford a compound of Formula F6:

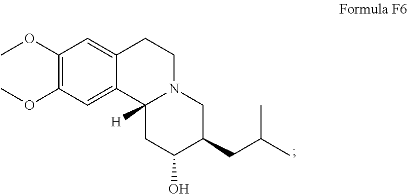 [SMILES: COc1cc2c(cc1OC)[C@H]1C[C@@H](O)[C@H](CC(C)C)CN1CC2]

f) coupling the compound of Formula F6 and a carboxylic acid of Formula F7:

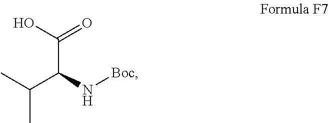

with N-(3-dimethylaminopropyl)-N′-ethylcarbodiimide hydrochloride (EDC·HCl) in the presence of 4-dimethylaminopyridine (DMAP) and dichloromethane to afford a compound of Formula F8:

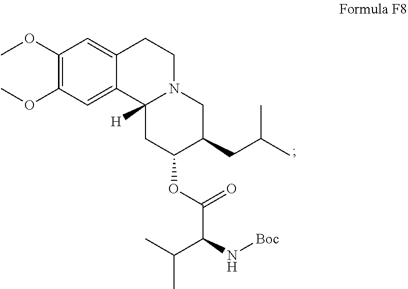 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) deprotecting the compound of Formula F8 with a mixture of hydrogen chloride and dioxane in the presence of dichloromethane to afford a compound of Formula F9-HCl:

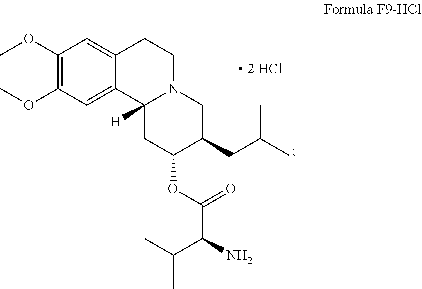 [SMILES: COc1cc2c(cc1OC)[C@H]1C[C@@H](OC(=O)[C@@H](N)C(C)C)[C@H](CC(C)C)CN1CC2]

h) reacting the compound of Formula F9-HCl with aqueous sodium bicarbonate in the presence of dichloromethane to afford a compound of Formula F9 (free base):

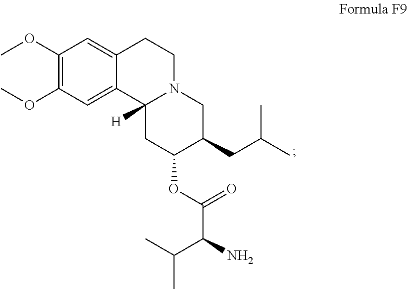 [SMILES: COc1cc2c(cc1OC)[C@H]1C[C@@H](OC(=O)[C@@H](N)C(C)C)[C@H](CC(C)C)CN1CC2]

and
i) reacting the compound of Formula F9 (free base) with p-toluenesulfonic acid in the presence of acetonitrile to afford the compound of Formula I.

In some embodiments, after Step g) and before Step h) the process further comprises the steps of:
1) reacting the compound of Formula F9-HCl with aqueous sodium bicarbonate in the presence of a solvent comprising dichloromethane to afford a compound of Formula F9 (free base):

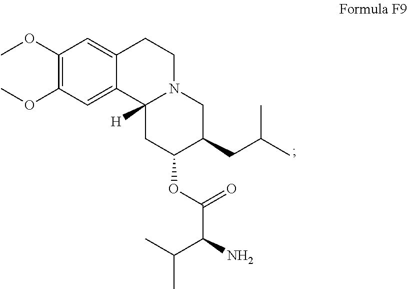 [SMILES: COc1cc2c(cc1OC)[C@H]1C[C@@H](OC(=O)[C@@H](N)C(C)C)[C@H](CC(C)C)CN1CC2]

and
2) reacting the compound of Formula F9 (free base) in a solvent comprising acetonitrile with a hydrogen chloride isopropanol mixture to afford the compound of Formula F9-HCl.

In some embodiments, after Step g) and before Step h) the process further comprises the steps of:
1) reacting the compound of Formula F9-HCl with aqueous sodium bicarbonate in the presence of a solvent comprising dichloromethane to afford a compound of Formula F9 (free base):

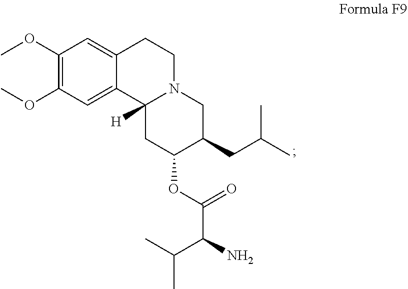 [SMILES: COc1cc2c(cc1OC)[C@H]1C[C@@H](OC(=O)[C@@H](N)C(C)C)[C@H](CC(C)C)CN1CC2]

and
2) reacting the compound of Formula F9 (free base) in a solvent comprising acetonitrile with a hydrogen chloride isopropanol mixture to afford the compound of Formula F9-HCl.

Another aspect of the present invention provides processes for the preparation of a compound of Formula I:

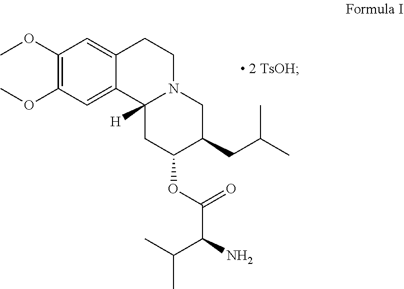 [SMILES: COc1cc2c(cc1OC)[C@H]1C[C@@H](OC(=O)[C@@H](N)C(C)C)[C@H](CC(C)C)CN1CC2]

comprising the steps of:
a) reacting a compound of Formula F1:

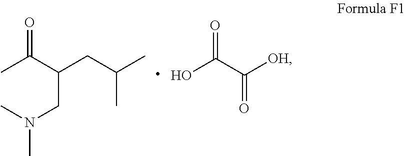

with aqueous potassium hydroxide in the presence of methyl tert-butyl ether (MTBE) to afford a compound of Formula F2:

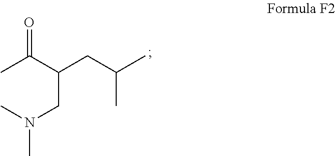

b) cyclizing the compound of Formula F2 with a compound of Formula F3:

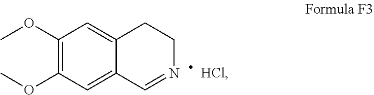

in the presence of sodium iodide, isopropanol (IPA), and water to afford a compound of Formula F4:

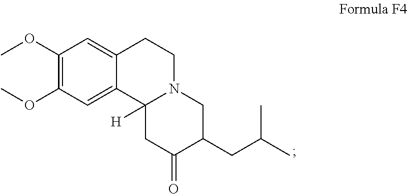 [SMILES: COc1cc2c(cc1OC)C1CC(=O)C(CC(C)C)CN1CC2]

c) reducing the compound of Formula F4 with sodium borohydride in the presence of methyl tert-butyl ether (MTBE), acetic acid, and methanol to afford a compound of Formula F5:

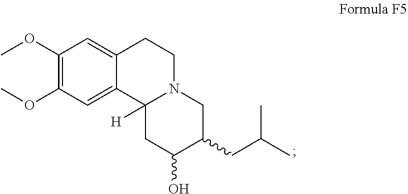 [SMILES: COc1cc2c(cc1OC)C1CC(O)C(CC(C)C)CN1CC2]

d) resolving the compound of Formula F5 with (S)-(+)-camphorsulfonic acid (CSA) in ethanol and water to afford a compound of Formula F6-CSA:

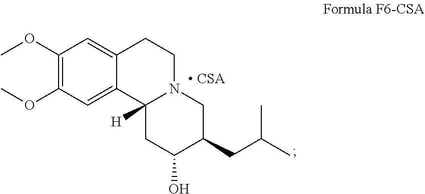 [SMILES: COc1cc2c(cc1OC)[C@H]1C[C@@H](O)[C@H](CC(C)C)CN1CC2]

e) reacting the compound of Formula F6-CSA with aqueous sodium hydroxide in dichloromethane to afford a compound of Formula F6:

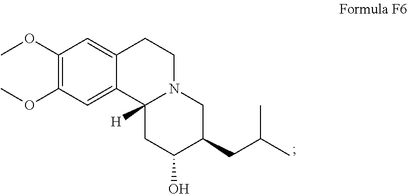 [SMILES: COc1cc2c(cc1OC)[C@H]1C[C@@H](O)[C@H](CC(C)C)CN1CC2]

f) coupling the compound of Formula F6 and a carboxylic acid of Formula F7:

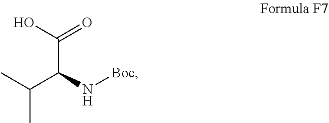

with N-(3-dimethylaminopropyl)-N′-ethylcarbodiimide hydrochloride (EDC·HCl) in the presence of 4-dimethylaminopyridine (DMAP) and dichloromethane to afford a compound of Formula F8:

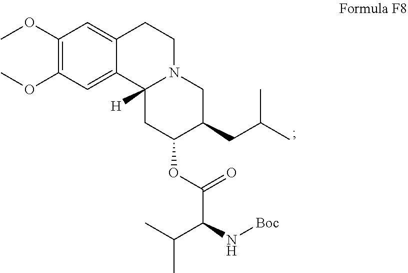 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) deprotecting the compound of Formula F8 with a mixture of hydrogen chloride and dioxane in the presence of dichloromethane to afford a compound of Formula F9-HCl:

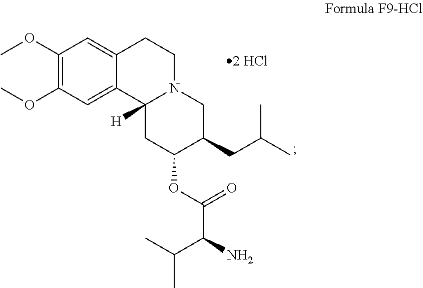 [SMILES: COc1cc2c(cc1OC)[C@H]1C[C@@H](OC(=O)[C@@H](N)C(C)C)[C@H](CC(C)C)CN1CC2]

reacting the compound of Formula F9-HCl with aqueous sodium bicarbonate in the presence of a solvent comprising dichloromethane to afford a compound of Formula F9 (free base):

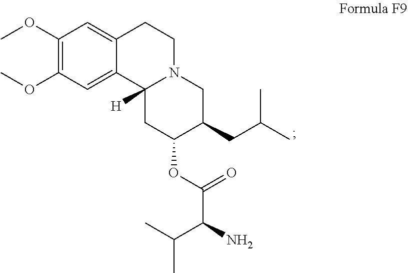 [SMILES: COc1cc2c(cc1OC)[C@H]1C[C@@H](OC(=O)[C@@H](N)C(C)C)[C@H](CC(C)C)CN1CC2]

reacting the compound of Formula F9 (free base) in a solvent comprising acetonitrile with a hydrogen chloride isopropanol mixture to afford the compound of Formula F9-HCl;
h) reacting the compound of Formula F9-HCl with aqueous sodium bicarbonate in the presence of dichloromethane to afford a compound of Formula F9 (free base); and
i) reacting the compound of Formula F9 with p-toluenesulfonic acid in the presence of acetonitrile to afford the compound of Formula I.

Another aspect of the present invention provides processes for the preparation of a compound of Formula I;

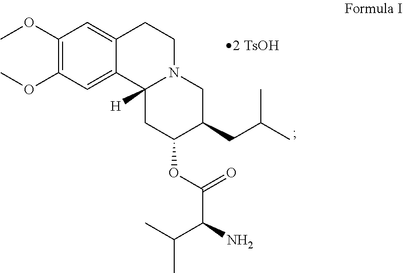 [SMILES: COc1cc2c(cc1OC)[C@H]1C[C@@H](OC(=O)[C@@H](N)C(C)C)[C@H](CC(C)C)CN1CC2]

comprising the steps of:
a) reacting a compound of Formula F1:

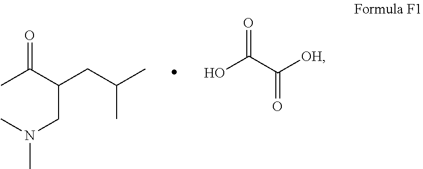

with aqueous potassium hydroxide in the presence of methyl tert-butyl ether (MTBE) to afford a compound of Formula F2:

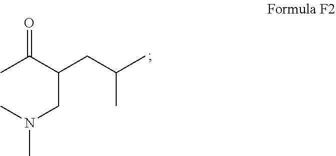

b) cyclizing the compound of Formula F2 with a compound of Formula F3:

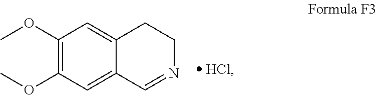

in the presence of sodium iodide, isopropanol (IPA), and water to afford a compound of Formula F4:

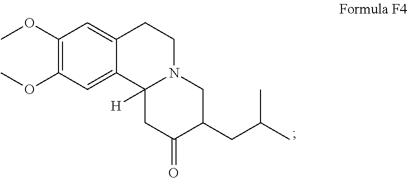 [SMILES: COc1cc2c(cc1OC)C1CC(=O)C(CC(C)C)CN1CC2]

c) reducing the compound of Formula F4 with sodium borohydride in the presence of methyl tert-butyl ether (MTBE), acetic acid, and methanol to afford a compound of Formula F5:

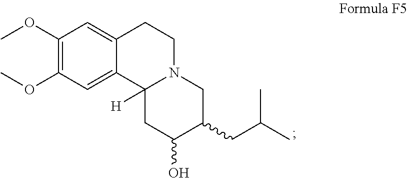 [SMILES: COc1cc2c(cc1OC)C1CC(O)C(CC(C)C)CN1CC2]

d) resolving the compound of Formula F5 with (S)-(+)-camphorsulfonic acid (CSA) in ethanol and water to afford a compound of Formula F6-CSA:

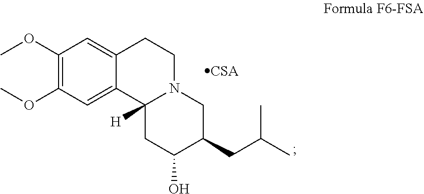 [SMILES: COc1cc2c(cc1OC)[C@H]1C[C@@H](O)[C@H](CC(C)C)CN1CC2]

e) reacting the compound of Formula F6-CSA with aqueous potassium hydroxide in 2-methyltetrahydrofuran (MeTHF) to afford a compound of Formula F6:

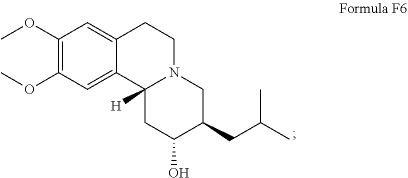 [SMILES: COc1cc2c(cc1OC)[C@H]1C[C@@H](O)[C@H](CC(C)C)CN1CC2]

f) coupling the compound of Formula F6 and a carboxylic acid of Formula F7:

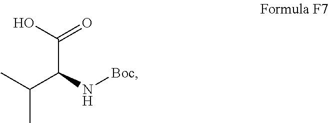

with N-(3-dimethylaminopropyl)-N′-ethylcarbodiimide (EDCI) in the presence of 4-dimethylaminopyridine (DMAP) and 2-methyltetrahydrofuran (MeTHF) to afford a compound of Formula F8:

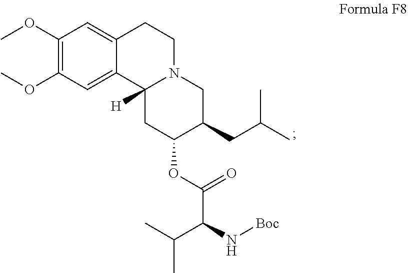 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) deprotecting the compound of Formula F8 with a mixture of hydrogen chloride and isopropanol in the presence of 2-methyltetrahydrofuran (MeTHF) and ethyl acetate (EtOAc) to afford a compound of Formula F9-HCl:

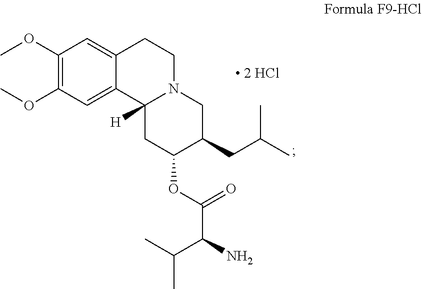 [SMILES: COc1cc2c(cc1OC)[C@H]1C[C@@H](OC(=O)[C@@H](N)C(C)C)[C@H](CC(C)C)CN1CC2]

h) reacting the compound of Formula F9-HCl with aqueous sodium bicarbonate in the presence of 2-methyltetrahydrofuran (MeTHF) and ethyl acetate (EtOAc) to afford a compound of Formula F9 (free base):

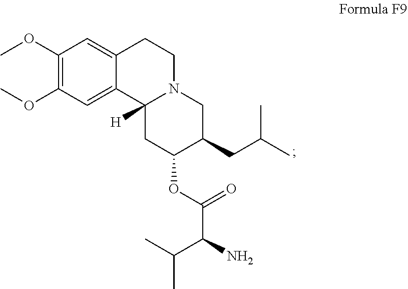 [SMILES: COc1cc2c(cc1OC)[C@H]1C[C@@H](OC(=O)[C@@H](N)C(C)C)[C@H](CC(C)C)CN1CC2]

and
i) reacting the compound of Formula F9 with p-toluenesulfonic acid in the presence of acetonitrile to afford the compound of Formula I.

Another aspect of the present invention provides processes for the preparation of a compound of Formula I;

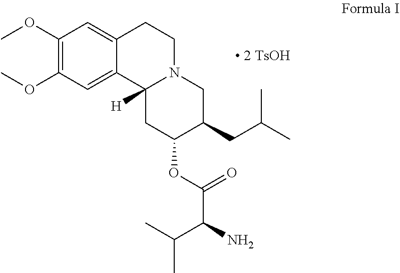 [SMILES: COc1cc2c(cc1OC)[C@H]1C[C@@H](OC(=O)[C@@H](N)C(C)C)[C@H](CC(C)C)CN1CC2]

comprising the steps of:
a) reacting a compound of Formula F1:

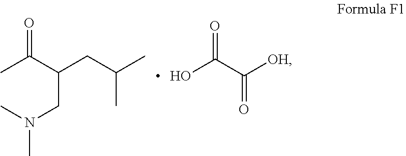

with aqueous potassium hydroxide in the presence of methyl tert-butyl ether (MTBE) to afford a compound of Formula F2:

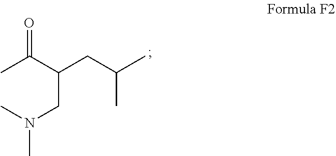

b) cyclizing the compound of Formula F2 with a compound of Formula F3:

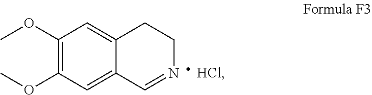

in the presence of sodium iodide, isopropanol (IPA), and water to afford a compound of Formula F4:

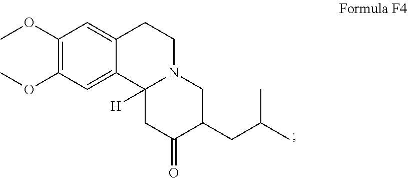 [SMILES: COc1cc2c(cc1OC)C1CC(=O)C(CC(C)C)CN1CC2]

c) reducing the compound of Formula F4 with sodium borohydride in the presence of methyl tert-butyl ether (MTBE), acetic acid, and methanol to afford a compound of Formula F5:

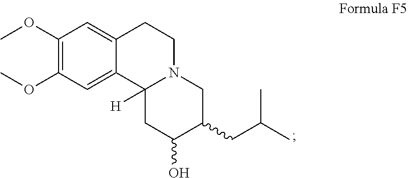 [SMILES: COc1cc2c(cc1OC)C1CC(O)C(CC(C)C)CN1CC2]

d) resolving the compound of Formula F5 with (S)-(+)-camphorsulfonic acid (CSA) in ethanol and water to afford a compound of Formula F6-CSA:

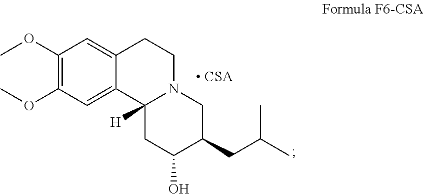 [SMILES: COc1cc2c(cc1OC)[C@H]1C[C@@H](O)[C@H](CC(C)C)CN1CC2]

e) reacting the compound of Formula F6-CSA with aqueous sodium hydroxide in dichloromethane to afford a compound of Formula F6:

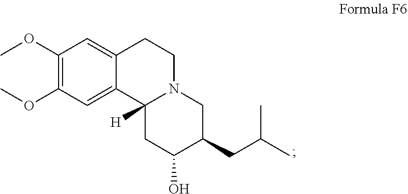 [SMILES: COc1cc2c(cc1OC)[C@H]1C[C@@H](O)[C@H](CC(C)C)CN1CC2]

f) coupling the compound of Formula F6 and a carboxylic acid of Formula F7:

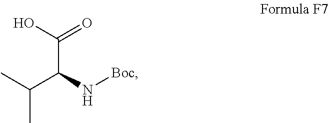

with N-(3-dimethylaminopropyl)-N′-ethylcarbodiimide hydrochloride (EDC·HCl) in the presence of 4-dimethylaminopyridine (DMAP) and dichloromethane to afford a compound of Formula F8:

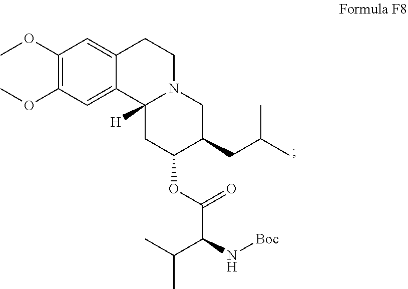 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) deprotecting the compound of Formula F8 with a mixture of hydrogen chloride and dioxane in the presence of dichloromethane to afford a compound of Formula F9-HCl:

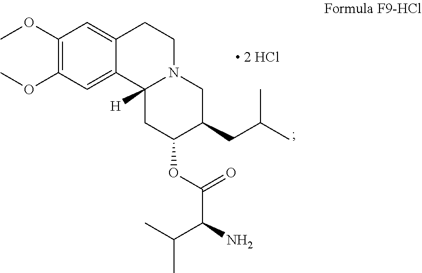 [SMILES: COc1cc2c(cc1OC)[C@H]1C[C@@H](OC(=O)[C@@H](N)C(C)C)[C@H](CC(C)C)CN1CC2]

and
h) reacting the compound of Formula F9-HCl with p-toluenesulfonic acid in a solvent comprising ethyl acetate (EtOAc) to afford the compound of Formula I.

In some embodiments, after Step g) and before Step h) the process further comprises the steps of:
1) reacting the compound of Formula F9-HCl with aqueous sodium bicarbonate in the presence of a solvent comprising dichloromethane to afford a compound of Formula F9 (free base):

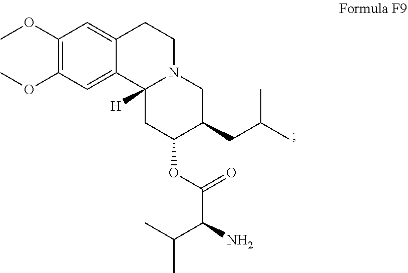 [SMILES: COc1cc2c(cc1OC)[C@H]1C[C@@H](OC(=O)[C@@H](N)C(C)C)[C@H](CC(C)C)CN1CC2]

and
2) reacting the compound of Formula F9 (free base) in a solvent comprising acetonitrile with a hydrogen chloride isopropanol mixture to afford the compound of Formula F9-HCl.

Another aspect of the present invention provides the compound of Formula I prepared by any of the processes as described herein.

Another aspect of the present invention provides processes for preparing a pharmaceutical composition comprising: preparing a compound of Formula I according to any of the processes described herein; and formulating the compound of Formula I with a pharmaceutically acceptable carrier and/or diluent.

In some embodiments, the pharmaceutical composition comprises: the compound of Formula I (i.e., valbenazine ditosylate); at least one water insoluble filler; at least one water soluble diluent; at least one binder; at least one disintegrant; and at least one lubricant. In some embodiments, the pharmaceutical composition comprises: the compound of Formula I having a w/w % of about 40%; at least one water insoluble filler having a w/w % of about 25%; at least one water soluble diluent having a w/w % of about 20%; at least one binder having a w/w % of about 5%; at least one disintegrant having a w/w % of about 7.5%; and at least one lubricant having a w/w % of about 2.5%.

In some embodiments, the pharmaceutically acceptable carrier and/or diluent in the pharmaceutical composition comprises silicified microcrystalline cellulose; isomalt; hydroxypropyl methylcellulose; partially pregelatinized maize starch; and magnesium stearate. In some embodiments, the pharmaceutical composition comprises: the compound of Formula I having a w/w % of about 40%; silicified microcrystalline cellulose having a w/w % of about 25%; isomalt having a w/w % of about 20%; hydroxypropyl methylcellulose having a w/w % of about 5%; partially pregelatinized maize starch having a w/w % of about 7.5%; and magnesium stearate having a w/w % of about 2.5%.

In some embodiments, the pharmaceutically acceptable carrier and/or diluent in the pharmaceutical composition comprises silicified microcrystalline cellulose; isomalt; hydroxypropyl methylcellulose; partially pregelatinized maize starch; and magnesium stearate.

Another aspect of the present invention provides processes for preparing a unit dosage form comprising: preparing a compound of Formula I according to any of the processes described herein; and formulating the compound of Formula I with a pharmaceutically acceptable carrier and/or diluent.

In some embodiments, the unit dosage form comprises: the compound of Formula I (i.e., valbenazine ditosylate); at least one water insoluble filler; at least one water soluble diluent; at least one binder; at least one disintegrant; and at least one lubricant. In some embodiments, the unit dosage form comprises: the compound of Formula I having a w/w % of about 40%; at least one water insoluble filler having a w/w % of about 25%; at least one water soluble diluent having a w/w % of about 20%; at least one binder having a w/w % of about 5%; at least one disintegrant having a w/w % of about 7.5%; and at least one lubricant having a w/w % of about 2.5%.

In some embodiments, the pharmaceutically acceptable carrier and/or diluent in the unit dosage form comprises silicified microcrystalline cellulose; isomalt; hydroxypropyl methylcellulose; partially pregelatinized maize starch; and magnesium stearate. In some embodiments, the unit dosage form comprises: the compound of Formula I having a w/w % of about 40%; silicified microcrystalline cellulose having a w/w % of about 25%; isomalt having a w/w % of about 20%; hydroxypropyl methylcellulose having a w/w % of about 5%; partially pregelatinized maize starch having a w/w % of about 7.5%; and magnesium stearate having a w/w % of about 2.5%.

In some embodiments, the pharmaceutically acceptable carrier and/or diluent in the unit dosage form comprises silicified microcrystalline cellulose; isomalt; hydroxypropyl methylcellulose; partially pregelatinized maize starch; and magnesium stearate.

In some embodiments, the compound of Formula I in the unit dosage form is present in an amount ranging from about 20 mg to 160 mg as measured as the free base (i.e., the compound of Formula F9). In some embodiments, the compound of Formula I in the unit dosage form is present in an amount of 20 mg, 40 mg, 60 mg, 80 mg, or 100 mg as measured as the free base. In some embodiments, the compound of Formula I in the unit dosage form is present in an amount of 40 mg, 60 mg, or 80 mg as measured as the free base. In some embodiments, the compound of Formula I in the unit dosage form is present in an amount of 20 mg as measured as the free base. In some embodiments, the compound of Formula I in the unit dosage form is present in an amount of 40 mg as measured as the free base. In some embodiments, the compound of Formula I in the unit dosage form is present in an amount of 60 mg as measured as the free base. In some embodiments, the compound of Formula I in the unit dosage form is present in an amount of 80 mg as measured as the free base. In some embodiments, the unit dosage form is suitable for oral administration. In some embodiments, the unit dosage form is formulated for a once daily dosing. In some embodiments, the unit dosage form is in a capsule form. In some embodiments, the capsule is size 1 or smaller. In some embodiments, the capsule is size 1, 2, or 3. In some embodiments, the capsule is size 1. In some embodiments, the capsule is size 2. In some embodiments, the capsule is size 3.

Some embodiments relate to pharmaceutical compositions prepared by any of the processes described herein.

Some embodiments relate to unit dosage forms prepared by any of the processes described herein.

Another aspect of the present application provides methods for inhibiting monoamine transporter isoform 2 (VMAT2) in a patient in need thereof, comprising administering to the patient a therapeutically effective amount of a pharmaceutical composition or a unit dosage form, wherein the pharmaceutical composition and the unit dosage form can be prepared according to any of the processes as described herein. Another aspect of the present application provides methods of treating a neurological or psychiatric disease or disorder in a patient in need thereof, comprising administering to the patient a therapeutically effective amount of a pharmaceutical composition or a unit dosage form, wherein the pharmaceutical composition and the unit dosage form can be prepared according to any of the processes as described herein. Another aspect of the present application provides methods of treating a hyperkinetic disorder in a patient in need thereof, comprising administering to the patient a therapeutically effective amount of a pharmaceutical composition or a unit dosage form, wherein the pharmaceutical composition and the unit dosage form can be prepared according to any of the processes as described herein.

Another aspect of the present application provides uses of a pharmaceutical composition or a unit dosage form for the manufacture of a medicament for inhibiting monoamine transporter isoform 2 (VMAT2) in a patient in need thereof, wherein the pharmaceutical composition and the unit dosage form can be prepared according to any of the processes as described herein. Another aspect of the present application provides uses of a pharmaceutical composition or a unit dosage form for the manufacture of a medicament for treating a neurological or psychiatric disease or disorder in a patient in need thereof, wherein the pharmaceutical composition and the unit dosage form can be prepared according to any of the processes as described herein. Another aspect of the present application provides uses of a pharmaceutical composition or a unit dosage form for the manufacture of a medicament for treating a hyperkinetic disorder in a patient in need thereof, wherein the pharmaceutical composition and the unit dosage form can be prepared according to any of the processes as described herein.

In some embodiments, the VMAT2 inhibitor is administered to the patient to treat a neurological or psychiatric disease or disorder. In some embodiments, the neurological or psychiatric disease or disorder is a hyperkinetic movement disorder, mood disorder, bipolar disorder, schizophrenia, schizoaffective disorder, mania in mood disorder, depression in mood disorder, treatment-refractory obsessive compulsive disorder, neurological dysfunction associated with Lesch-Nyhan syndrome, agitation associated with Alzheimer's disease, Fragile X syndrome or Fragile X-associated tremor-ataxia syndrome, autism spectrum disorder, Rett syndrome, or chorea-acanthocytosis. In some embodiments, the neurological or psychiatric disease or disorder is in a patient with intellectual and developmental disability (IDD).

In some embodiments, the neurological or psychiatric disease or disorder is a hyperkinetic movement disorder. In some embodiments, the hyperkinetic movement disorder is tardive dyskinesia. In some embodiments, the hyperkinetic movement disorder is a tic disorder. In some embodiments, the tic disorder is Tourette's Syndrome. In some embodiments, the hyperkinetic movement disorder is Huntington's disease. In some embodiments, the hyperkinetic movement disorder is choreiform movements, general dystonia, focal dystonia, and myoclonus movements. In some embodiments, the hyperkinetic movement disorder is chorea associated with Huntington's disease. In some embodiments, the hyperkinetic movement disorder is ataxia, chorea, dystonia,

Huntington's disease, myoclonus, restless leg syndrome, or tremors. In some embodiments, the hyperkinetic movement disorder is a disease or disorder other than Huntington's disease. In some embodiments, the hyperkinetic movement disorder described herein is not in a patient with intellectual and developmental disability (IDD). In some embodiments, the hyperkinetic movement disorder described herein is in a patient with intellectual and developmental disability (IDD), for example, in some embodiments, the hyperkinetic movement disorder is tardive dyskinesia in a patient with intellectual and developmental disability (IDD).

### Definitions

For clarity and consistency, the following definitions will be used throughout this patent document.

As used in the specification and the accompanying claims, the indefinite articles “a” and “an” and the definite article “the” include plural as well as singular referents, unless the context clearly dictates otherwise.

The term “about” or “approximately.” means an acceptable error for a particular value as determined by one of ordinary skill in the art, which depends in part on how the value is measured or determined. In some embodiments, the term “about” or “approximately” means within 1, 2, 3, or 4 standard deviations. In some embodiments, the term “about” or “approximately” means within 30%, 25%, 20%, 15%, 10%, 9%, 8%, 7%, 6%, 5%, 4%, 3%, 2%, 1%, 0.5% or 0.05% of a given value or range.

The term “crystalline form” of a compound refers to any crystalline form of the compound as a free acid, the compound as a free base, as an acid addition salt of the compound, a base addition salt of the compound, a complex of the compound, a solvate (including hydrate) of the compound, or a co-crystal of the compound. The term “solid form” of a compound can refer to any crystalline form of the compound or any amorphous form of the compound as a free acid, the compound as a free base, as an acid addition salt of the compound, an base addition salt of the compound, a complex of the compound, or a solvate (including hydrate) of the compound, or a co-precipitation of the compound. In many instances, the terms “crystalline form” and “solid form” can refer to those that are pharmaceutically acceptable, including, for example, those of pharmaceutically acceptable addition salts, pharmaceutically acceptable complexes, pharmaceutically acceptable solvates, pharmaceutically acceptable co-crystals, and pharmaceutically acceptable co-precipitations.

The terms “process” and “method” are used interchangeably to refer to a method disclosed herein for a compound preparation. Modifications to the processes and methods disclosed herein (e.g., starting materials, reagents, protecting groups, solvents, temperatures, reaction times, and/or purification) that are well known to those of ordinary skill in the art are also encompassed by the disclosure.

The terms “adding”, “reacting” and “mixing” are used interchangeably to refer to contacting one reactant, reagent, solvent, catalyst, or a reactive group with another reactant, reagent, solvent, catalyst, or reactive group. Unless otherwise specified, reactants, reagents, solvents, catalysts, and reactive groups can be added individually, simultaneously, or separately, and/or can be added in any order. They can be added in the presence or absence of heat, and can optionally be added under an inert atmosphere (e.g., N₂ or Ar). In some embodiments, the term “reacting” can also refer to in situ formation or intra-molecular reaction where the reactive groups are in the same molecule.

The term “substantially anhydrous” refers to a solution, mixture, solid (crystalline, or amorphous, or mixtures thereof), and the like, that has a % water content of 5% or less, 4% or less, 3% or less, 2% or less, 1% or less, 0.5% or less, 0.1% or less, at the limit of detection, or below the limit of detection, as determined by an analytical method known in the art, such as, a Karl Fischer Titrator, and the like.

The present application also includes salts of the compounds described herein. As used herein, “salts” refers to derivatives of the disclosed compounds wherein the parent compound is modified by converting an existing acid or base moiety to its salt form. Examples of salts include, but are not limited to, mineral acid (such as HCl, HBr, H₂SO₄) or organic acid (such as acetic acid, benzoic acid, trifluoroacetic acid) salts of basic residues such as amines; alkali (such as Li, Na, K, Mg. Ca) or organic (such as trialkylammonium) salts of acidic residues such as carboxylic acids; and the like. The salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as High Performance Liquid Chromatography (HPLC) or thin layer chromatography. The compounds obtained by the reactions can be purified by any suitable method known in the art. For example, chromatography (medium pressure) on a suitable adsorbent (e.g., silica gel, alumina, and the like), HPLC, or preparative thin layer chromatography; distillation; sublimation, trituration, or recrystallization. The purity of the compounds, in general, are determined by physical methods such as measuring the melting point (in case of a solid), obtaining an NMR spectrum, or performing a HPLC separation. If the melting point decreases, if unwanted signals in the NMR spectrum are decreased, or if extraneous peaks in an HPLC trace are removed, the compound can be said to have been purified. In some embodiments, the compounds are substantially purified.

The reactions of the processes described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the reaction step, suitable solvent(s) for that particular reaction step can be selected. Appropriate solvents include water, alkanes (such as pentanes, hexanes, heptanes, cyclohexane, etc., or a mixture thereof), aromatic solvents (such as benzene, toluene, xylene, etc.), alcohols (such as methanol, ethanol, isopropanol, etc.), ethers (such as dialkylethers, methyl tert-butyl ether (MTBE); substituted and unsubstituted cycloalkyl ethers, 2-methyltetrahydrofuran (MeTHF), tetrahydrofuran (THF), dioxane, etc.), esters (such as ethyl acetate, butyl acetate, etc.), halogenated hydrocarbon solvents (such as dichloromethane (DCM), chloroform, dichloroethane, tetrachloroethane), dimethylformamide (DMF), dimethyl sulfoxide (DMSO), acetone, acetonitrile (ACN), hexamethylphosphoramide (HMPA) and N-methyl pyrrolidone (NMP). Such solvents can be used in either their wet or anhydrous forms.

Crystals used for seeding can be obtained from the previous syntheses, see for example, PCT publications WO2017/112857 and WO2021/050977.

### EXAMPLES

The disclosure will be described in greater detail by way of specific examples. The following examples are offered for illustrative purposes and are not intended to limit the disclosure in any manner. Those of skill in the art will readily recognize a variety of non-critical parameters which can be changed or modified to yield essentially the same results. Unless otherwise specified, the reactions set forth below were done generally at ambient temperature or room temperature. Reactions were assayed by HPLC, and terminated as judged by the consumption of starting material.

The compound structures and purities in the examples below were confirmed by one or more of the following methods: proton nuclear magnetic resonance (¹H NMR) spectroscopy, ¹³C NMR spectroscopy, mass spectroscopy, infrared spectroscopy, melting point, X-ray crystallography, and/or HPLC. ¹H NMR spectra were determined using an NMR spectrometer operating at a certain field strength. Chemical shifts are reported in parts per million (ppm, δ) downfield from a standard, e.g., an internal standard, such as TMS. Alternatively, 1H NMR spectra were referenced to signals from residual protons in deuterated solvents as follows: CDCl₃=7.26 ppm; DMSOd₆=2.50 ppm; C₆D₆=7.16 ppm; CD₃OD=3.31 ppm (J. Org. Chem. 1997, 62, 7513). Peak multiplicities are designated as follows; s, singlet; d, doublet; dd, doublet of doublets; t, triplet; dt, doublet of triplets; q, quartet; br, broadened; and m, multiplet. Coupling constants are given in Hertz (Hz). Mass spectra (MS) data were obtained using a mass spectrometer with APCI or ESI ionization.

The compounds described herein, supra and infra, are named according to MarvinSketch 18.24.0 or ChemDraw Professional 18.2.0.48. In certain instances, when common names are used it is understood that these common names would be recognized by those skilled in the art.

### Example 1: Synthesis of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I)

### Step A: Synthesis of 3-Isobutyl-9,10-dimethoxy-3,4,6,7-tetrahydro-1H-pyrido[2,1-a]isoquinolin-2(11bH)-one

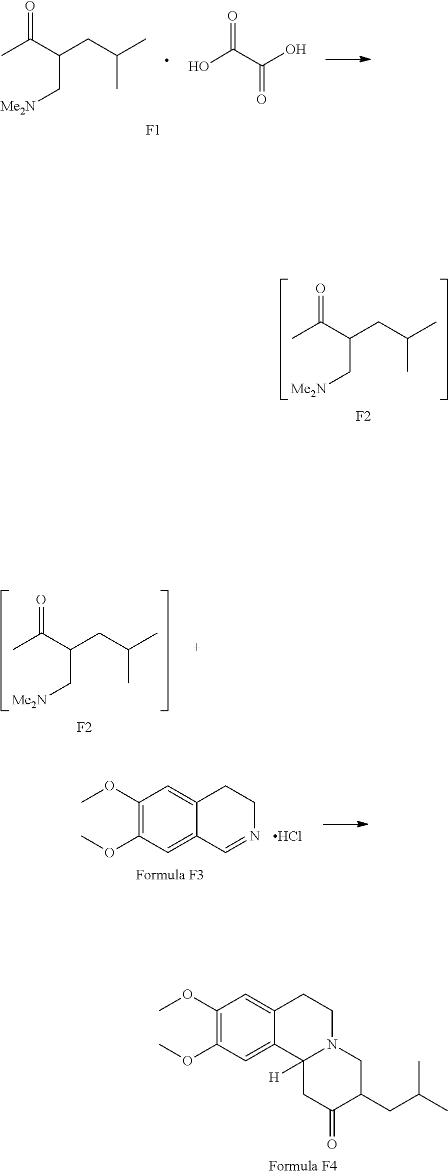

To a reactor was charged demineralized water (231 L, 6.30 V), 3-((dimethylamino)methyl)-5-methylhexan-2-one oxalate (Formula F1, 52.6 kg, 202 mol: 1.25 equiv.) and methyl tert-butyl ether (95 L, 2.60 V). The resulting mixture was heated to about 22° C. and the pH adjusted to 11 with 10 wt % potassium hydroxide solution (210.8 kg, 376 mol, 2.33 equiv.) and stirred for no less than (“NLT”) 15 min. The resulting layers were split, and the organic layer containing the free base (Formula F2) was washed with demineralized water (39 L, 1.05 V). The solvent was exchanged by put and take distillation at 1.50 V with isopropanol (129 L, 3.50 V). The mixture was cooled to about 22° C. (19 to 25° C.) and charged with demineralized water (55 L, 1.50 V), sodium iodide (9.7 kg, 65 mol, 0.40 equiv.), and 6,7-dimethoxy-3,4-dihydroisoquinoline hydrochloride (Formula F3, 36.7 kg, 161 mmol, 1.00 equiv.) and heated to about 42° C. with stirring for NLT 24h. The mixture was cooled to about 22° C. and stirred for NLT 1h. The resulting solid was isolated by filtration and the filter cake washed with isopropanol (91.8

L, 2.50 V). The isolated solid was dried at about 40° C. under vacuum for NLT 12 h to afford 3-isobutyl-9,10-dimethoxy-3,4,6,7-tetrahydro-1H-pyrido[2,1-a]isoquinolin-2(11bH)-one (Formula F4). Yield: 45.3 kg, 143 mol, 88.5%, with 99.2% purity.

### Step B: Synthesis of 3-Isobutyl-9,10-dimethoxy-2,3,4,6,7,11b-hexahydro-1H-pyrido[2,1-a]isoquinolin-2-ol

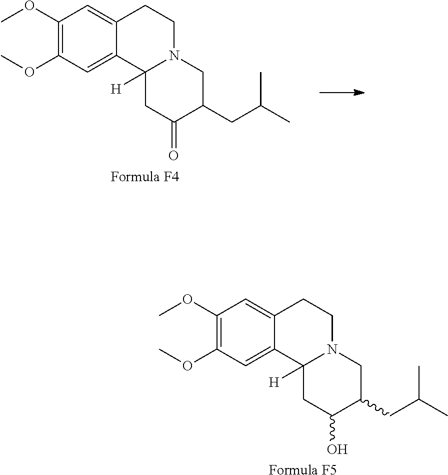

To a reactor was charged 3-isobutyl-9, 10-dimethoxy-3,4,6,7-tetrahydro-1H-pyrido[2,1-a]isoquinolin-2(11bH)-one (Formula F4, 44.3 kg, 139 mol, 1.00 equiv.), methyl tert-butyl ether (195 L, 4.40 V), acetic acid (9.3 kg, 155 mol, 1.11 equiv.), and methanol (44 L, 1.00 V). To the mixture was charged a suspension of sodium borohydride (10.5 kg, 279 mol, 2.00 equiv) in methyl tert-butyl ether (44 L, 1.00 V) keeping the temperature at about 22° C. The preparation vessel and the transfer line were rinsed with methyl tert-butyl ether (2×13 L, 2×0.30 V). The resulting mixture was stirred at about 25° C. for 2 h and a 1 N sodium hydroxide solution (230 kg, 222 mol, 1.59 equiv) was added (about 25° C.). The mixture was heated to about 47° C. with stirring (about 3 h) and cooled to about 15° C. with stirring (about 30 min). The resulting solid was isolated by filtration. The filter cake was washed with water (4×44 L, 4×1.00 V) and methyl tert-butyl ether (44 L, 1.00 V), and dried at about 40° C. under vacuum for NLT 12 h to afford 3-isobutyl-9,10-dimethoxy-2,3,4,6,7,11b-hexahydro-1H-pyrido[2,1-a]isoquinolin-2-ol (Formula F5). Yield: 35.6 kg, 111 mol, 80.1% with 99.0% purity

### Step C: Synthesis of (2R,3R,11bR)-3-Isobutyl-9,10-dimethoxy-2,3,4,6,7,11b-hexahydro-1H-pyrido[2,1-a]isoquinolin-2-ol (S)-(+)-camphorsulfonate

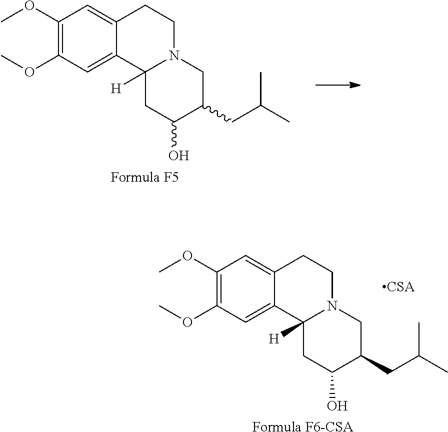

To a reactor was charged absolute ethanol (428 L, 12.00 V), camphor D-(+)-sulfonic acid (21.4 kg, 92 mol, 0.825 equiv), 3-isobutyl-9,10-dimethoxy-2,3,4,6,7,11b-hexahydro-1H-pyrido[2,1-a]isoquinolin-2-ol (Formula F5, 35.7 kg, 112 mol, 1.00 equiv), and demineralized water (0.75 V). The mixture was heated to about 70° C. with stirring (about 30 min) and cooled to about 22° C. at about 3 ° C/h and stirred for about 2 h. If the product had not crystallized, then a seed crystal of F6 CSA (0.2 kg, 0.5 wt %) was added. The resulting crystalline solid was isolated by filtration. The filter cake was washed with absolute ethanol (36 L, 1.00 V) and dried at about 45° C. under vacuum for NLT 12 h to afford (2R,3R,11bR)-3-isobutyl-9,10-dimethoxy-2,3,4,6,7,11b-hexahydro-1H-pyrido[2,1-a]isoquinolin-2-ol (S)-(+)-camphorsulfonate (Formula F6-CSA). Yield: 23.0 kg, 42 mol, 37.3% with 99.6% purity.

### Step D: Synthesis of (S)-(2R,3R,11bR)-3-Isobutyl-9,10-dimethoxy-2,3,4,6,7,11b-hexahydro-1H-pyrido[2,1-a]isoquinolin-2-yl 2-((tert-Butoxycarbonyl)amino)-3-methylbutanoate

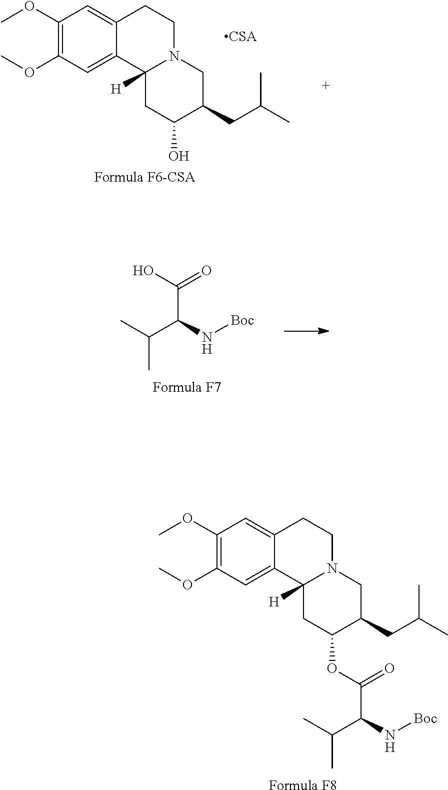

(2R,3R, 1 1bR)-3-isobutyl-9,10-dimethoxy-2,3,4,6,7,11b-hexahydro-1H-pyrido[2,1-a]isoquinolin-2-ol (1S)-(+)-camphorsulfonate (25.9 kg) was dissolved in dichloromethane (129.5 L, 5 volumes) and 1N sodium hydroxide (11.1 kg dissolved in 282.2 L of water) (pH>10), and then the mixture was stirred at 25±5° C. The organics were collected and washed with additional sodium hydroxide solution, and then with water. The organic phase was collected, dried with sodium sulfate, and then filtered to remove the solids. Boc-L-valine (12.2 kg, 1.2 equivalents) and 4-dimethylaminopyridine (1.55 kg, 0.3 equivalents) were charged to the organic phase and the mixture was then cooled to approximately 0° C. N-(3-dimethylaminopropyl)-N′ -ethylcarbodiimide hydrochloride (15.8 kg, 1.8 equivalents) was charged and the reaction was stirred for >3 hours. The reaction mixture was kept at 0±5° C. and was monitored by HPLC for completion. Once complete, water was added, and the contents were agitated. After settling, the water layer was discharged. The organic layer was washed with aqueous citric acid (prepared from 5.2 kg citric acid in 101 L of water) and then with water, to yield (S)-(2R,3R, 11bR)-3-isobutyl-9,10-dimethoxy-2,3,4,6,7,11b-hexahydro-1H-pyrido[2,1-a]isoquinolin-2-yl 2-((tert-butoxycarbonyl)amino)-3-methylbutanoate as a solution in dichloromethane.

### Step E: Synthesis of (S)-(2R,3R,11bR)-3-Isobutyl-9,10-dimethoxy-2,3,4,6,7,11b-hexahydro-1H-pyrido[2,1-a]isoquinolin-2-yl 2-Amino-3-methylbutanoate dihydrochloride

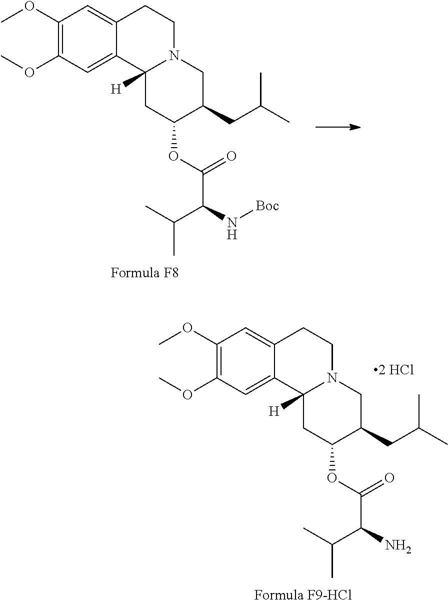

Hydrogen chloride in dioxane (4M, 57 L, 5 equivalents) was slowly added to a solution of (S)-(2R,3R,11bR)-3-isobutyl-9,10-dimethoxy-2,3,4,6,7,11b-hexahydro-1H-pyrido[2,1-a]isoquinolin-2-yl 2-((tert-butoxycarbonyl)amino)-3-methylbutanoate in dichloromethane, while maintaining the temperature between 5-10° C. Once the addition was complete, the mixture was agitated at 25±5° C. for >12 hours. Upon completion, aqueous sodium bicarbonate (217.6 kg) was slowly added and the mixture was agitated at 25±5° C. until pH>7. The organics were collected and washed with additional aqueous sodium bicarbonate, and then with water. Sodium sulfate was added to the organic layer and the mixture was then filtered to remove the solids. The organic layer was then distilled to the minimum volume required for agitation. Acetonitrile (70 L) was added and the mixture was again distilled down to minimum volume. Acetonitrile was added until the solution was a total of 10 volumes, and then the solution was cooled to 10±5° C. Hydrogen chloride in isopropanol (3.7 M, 26.4 L, 2.1 equivalents) was slowly added, followed by ethyl acetate (57 L) and the mixture was then heated to 50±5° C. Additional ethyl acetate was added followed by (S)-(2R,3R, 11bR)-3-isobutyl-9,10-dimethoxy-2,3,4,6,7,11b-hexahydro-1H-pyrido[2,1-a]isoquinolin-2-yl 2-amino-3-methylbutanoate dihydrochloride seeds and the mixture was heated to 75±5° C.for >1 hour. The slurry was slowly cooled to 25±5° C., and the solids were filtered, washed with ethyl acetate, and then dried under vacuum to yield (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate dihydrochloride (16.8 kg, 73% yield). Another batch was carried out starting from (S)-(2R,3R, 1 1bR)-3-isobutyl-9,10-dimethoxy-2,3,4,6,7,11b-hexahydro-1H-pyrido[2,1-a]isoquinolin-2-yl 2-amino-3-methylbutanoate (24.4 kg), using the same procedure described herein, to give (S)-(2R,3R, 11bR)-3-isobutyl-9,10-dimethoxy-2,3,4,6,7,11b-hexahydro-1H-pyrido[2,1-a]isoquinolin-2-yl 2-amino-3-methylbutanoate dihydrochloride (17 kg, 79% yield).

### Step F (Method 1): Synthesis of (S)-(2R,3R,11bR)-3-Isobutyl-9,10-dimethoxy-2,3,4,6,7,11b-hexahydro-1/-pyrido[2,1-a]isoquinolin-2-yl 2-amino-3-methylbutanoate di(4-methylbenzenesulfonate)

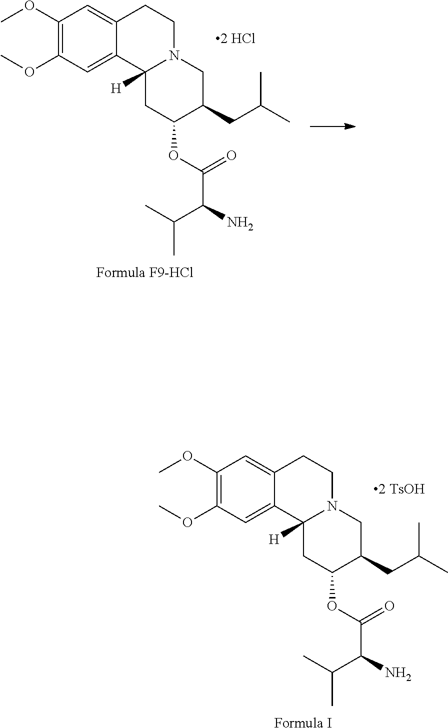

(S)-(2R,3R, 11bR)-3-isobutyl-9,10-dimethoxy-2,3,4,6,7,11b-hexahydro-1H-pyrido[2, 1-a]isoquinolin-2-yl 2-amino-3-methylbutanoate dihydrochloride (10.2 kg) was dissolved in dichloromethane (9 volumes) and aqueous sodium bicarbonate. The mixture was stirred at about 25° C. The organics were collected and washed with additional aqueous sodium bicarbonate, and then washed with water. The organic layer was collected, and acetonitrile added to the dichloromethane solution. The solution was distilled to the minimum volume required for stirring. Additional acetonitrile was added, and the mixture was distilled down to minimum volume. The mixture was tested for moisture content, then warmed to about 50° C. To this mixture, a solution of p-toluenesulfonic acid (2 equivalents) in acetonitrile was slowly added and the contents were agitated for >8 hours at about 50° C. The slurry was then cooled to about 25° C. and the solids filtered, washed with acetonitrile, and then dried under vacuum to yield (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (14.7 kg, 92.8% yield, 99.9% pure).

### Step F (Method 2): Synthesis of (S)-(2R,3R,11bR)-3-Isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate)

(S)-(2R,3R,1 1bR)-3-isobutyl-9, 10-dimethoxy-2,3,4,6,7,11b-hexahydro-1H-pyrido[2,1-a]isoquinolin-2-yl 2-amino-3-methylbutanoate dihydrochloride (15 kg) was suspended in dichloromethane (136.5 L, 9 volumes), aqueous sodium bicarbonate (245 kg) was added until pH >6.5, and then the mixture was agitated at 25±5° C. The organics were collected and washed with additional aqueous sodium bicarbonate, and then washed with water. The solution was then distilled to the minimum volume required for agitation. Acetonitrile (54 L) was added and the mixture was distilled down to minimum volume and repeated. Acetonitrile was added and the mixture was tested for moisture content and, once within the specification it was warmed to 50 +5° C. To this mixture, a solution of p-toluenesulfonic acid (11.7 kg, 2 equivalents) in acetonitrile (55.5 L) was slowly added and the contents were agitated for >8 hours at 50±5° C. The slurry was then cooled to 25±5° C. and the solids were filtered, washed with acetonitrile, and then dried under vacuum to yield (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (20.6 kg, 88% yield, ≥ 98% pure).

### Example 2: Preparation of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I)

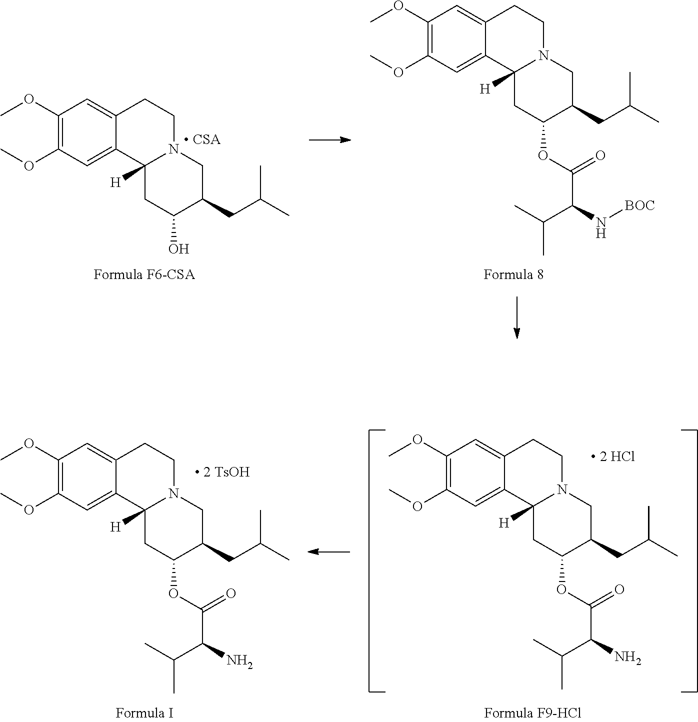

To an Erlenmeyer flask was charged (2R,3R,11bR)-3-isobutyl-9,10-dimethoxy-2,3,4,6,7,11b-hexahydro-1H-pyrido[2,1-a]isoquinolin-2-ol (1S)-(+)-camphorsulfonate (20 g) in 2-methyltetrahydrofuran (MeTHF) (100 mL), followed by aqueous KOH (2 M, 110 mL). The mixture was agitated for 15 min. The resulting biphasic solution was transferred to a separatory funnel and the layers were allowed to separate. An emulsion layer formed which was broken up with brine for better separation. The aqueous layer was discarded. To the organic layer was added H₂O (20 mL) followed by shaking several times. After 15 min, the layers were separated, and the aqueous layer was discarded.

To a round bottom flask was added the MeTHF solution of the free based material (˜100 mL; from above) along with additional MeTHF (40 mL). N-Boc-(L)-Val-OH (1.2 eq.) and DMAP (0.27 eq.) were added, after which a clear yellow solution resulted. The solution was cooled to 0 to −10° C. with an acetone ice/H2O bath. After reaching temperature, 1-ethyl-3-(3-dimethylaminopropyl) carbodiimide (EDCI) (1.77 eq.) was added and stirring was continued at 0 to −10° C. for 3 h. After 3 h, the ice bath was removed, and the reaction was agitated for at least 5 h. The analytical data indicated complete conversion to (S)-(2R,3R,11bR)-3-isobutyl-9,10-dimethoxy-2,3,4,6,7,11b-hexahydro-1H-pyrido[2,1-a]isoquinolin-2-yl 2-((tert-butoxycarbonyl)amino)-3-methylbutanoate after 18 h. The reaction was quenched with 5% aqueous citric acid (78 mL) followed by washing the organic layer with H₂O (60 mL). The resulting organic solution consisted of (S)-(2R,3R,11bR)-3-isobutyl-9,10-dimethoxy-2,3,4,6,7,11b-hexahydro-1H-pyrido[2,1-a]isoquinolin-2-yl 2-((tert-butoxycarbonyl)amino)-3-methylbutanoate which was carried onto the deprotection step without further purification. In an alternative procedure, (S)-(2R,3R,11bR)-3-isobutyl-9,10-dimethoxy-2,3,4,6,7,11b-hexahydro-1H-pyrido[2,1 -a]isoquinolin-2-yl 2-((tert-butoxycarbonyl)amino)-3-methylbutanoate was isolated by evaporating the organic solution.

The (S)-(2R,3R,11bR)-3-isobutyl-9,10-dimethoxy-2,3,4,6,7,11b-hexahydro-1H-pyrido[2,1-a]isoquinolin-2-yl 2-((tert-butoxycarbonyl)amino)-3-methylbutanoate solution from above was transferred to a clean round bottom flask, along with additional MeTHF (110 mL). To the solution was added EtOAc (44 mL) and 3.7 N HCl/isopropanol (21 mL: other HCl solutions can be used). The solution was heated to 45° C., seeded with (S)-(2R,3R,11bR)-3-isobutyl-9,10-dimethoxy-dihydrochloride, 2,3,4,6,7,11b-hexahydro-1H-pyrido[2,1-a]isoquinolin-2-yl 2-amino-3 -methylbutanoate and stirred for ½ h. After ½h, more EtOAc (30 mL) was added and the temperature was increased to 70° C. for 1 h. After 1 h, HPLC showed that 8% starting material still remained. To the reaction was added more 3.7 N HCl/isopropanol (3 mL), followed by heating at 70° C. for 2 h. After 2 h, the reaction was complete. Saturated aqueous NaHCO₃ (30 mL) was slowly added and the mixture was stirred for ½ h and was then washed with H₂O (60 mL). The resulting solution of freebased material (HPLC >95% purity) was carried onto the tosylate salt formation without further purification.

The free base solution from above was evaporated and a solvent exchange was completed with acetonitrile (2×40 mL). The yellow residue was dissolved in acetonitrile (67 mL) and heated to 45-55° C., after which a solution of p-TsOH/acetonitrile (8.3 g/139 mL) was added in one portion. After stirring for 18 h at 45° C., the slurry was cooled to 25° C., the white solid was filtered and washed with EtOAc (2×10 mL), and then dried in a vacuum oven at 50° C. for 18 h to afford (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (14.5 g, 53% overall isolated yield). The analytical HPLC data confirmed purity (99.68%) and chirality (99.77%).

In an alternative procedure, (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate dihydrochloride was isolated by filtration before freebasing and then converted to the ditosylate salt as described above.

### Example 3: Preparation of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate)

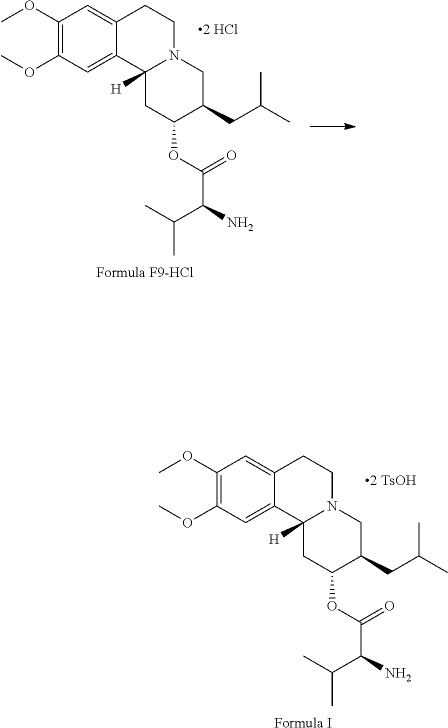

Isolated (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate dihydrochloride (10 g, 0.02 mol) was suspended in EtOAc (500 mL) and was then heated to 70° C. As the mixture was heating, p-TsOH (14 g, 4 eq.) was added. During heating, the mixture became a clear homogenous solution. The solution was aged for 2-3 h at 70° C. After 2-3 h, a white solid precipitated, and the heating source was removed. The suspension was stirred for 18 h and was then filtered. The solid was washed with EtOAc, and then dried in a vacuum oven at 50° C. for 18 h to afford (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (13.2 g, 88% isolated yield) as a white solid. The 1H-NMR of the sample matched the one obtained from Example 2.

### Example 4: p-Toluenesulfonic Acid Determination

The determination for the % area of p-toluenesulfonic acid in a sample of the compound of Formula I can be determined using the reverse-phase HPLC method as described in WO2021/050977.

### Example 5: Preparation of Capsules Containing 40 mg and 80 mg Valbenazine

Capsules containing 40 mg and 80 mg valbenazine (measured as the free base) can be prepared according to the methods described in WO2019/060322, incorporated herein by reference in its entirety.

The ingredients for exemplary 40 mg capsules are provided in the Table 1 below.

TABLE 1
40 mg Valbenazine Capsule (Size 2)

| Component | Function | Weight (mg/unit) | % (w/w) |
|---|---|---|---|
| Valbenazine ditosylate | Drug | 72.90 | 40.0 |
| (compound of Formula I) | Substance | (equivalent to 40 mg | |
| | | Free Base) | |
| Silicified microcrystalline | Diluent | 45.62 | 25.0 |
| cellulose | | | |
| Isomalt | Diluent | 36.50 | 20.0 |
| Hypromellose | Binder | 9.13 | 5.0 |
| Partially pregelatinized | Disintegrant | 13.69 | 7.5 |
| maize starch | | | |
| Magnesium stearate | Lubricant | 4.56 | 2.5 |
| Total Weight | | 182.40 | 100.00 |

The ingredients for exemplary 80 mg capsules are provided in the Table 2 below.

TABLE 2
80 mg Valbenazine Capsule (Size 1)

| Component | Function | Weight (mg/unit) | % (w/w) |
|---|---|---|---|
| Valbenazine ditosylate | Drug | 145.80 | 40.0 |
| (compound of Formula I) | Substance | (equivalent to 80 mg | |
| | | Free Base) | |
| Silicified microcrystalline | Diluent | 91.25 | 25.0 |
| cellulose | | | |
| Isomalt | Diluent | 73.00 | 20.0 |
| Hypromellose | Binder | 18.25 | 5.0 |
| Partially pregelatinized | Disintegrant | 27.38 | 7.5 |
| maize starch | | | |
| Magnesium stearate | Lubricant | 9.12 | 2.5 |
| Total Weight | | 364.80 | 100.00 |

Various modifications of the embodiments, in addition to those described herein, will be apparent to those skilled in the art from the foregoing description. Such modifications are also intended to fall within the scope of the appended claims. Each reference, including all patents, patent applications, and publications, cited in the present application is incorporated herein by reference in its entirety.

1. A process for preparing a compound of Formula I: 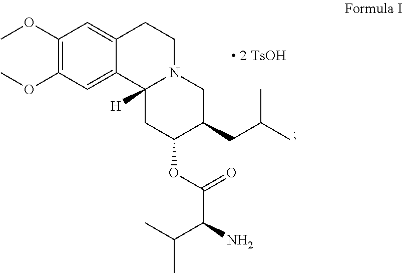 [SMILES: COc1cc2c(cc1OC)[C@H]1C[C@@H](OC(=O)[C@@H](N)C(C)C)[C@H](CC(C)C)CN1CC2]
comprising the steps of:
a) reacting a compound of Formula F1: 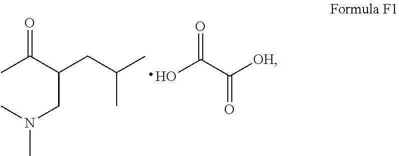
with a Step a)-base to afford a compound of Formula F2: 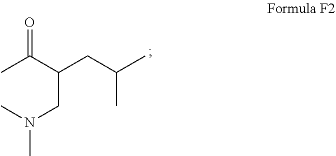
b) cyclizing the compound of Formula F2 with a compound of Formula F3: 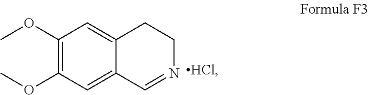
in the presence of sodium iodide to afford a compound of Formula F4: 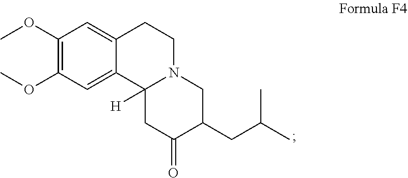 [SMILES: COc1cc2c(cc1OC)C1CC(=O)C(CC(C)C)CN1CC2]
c) reducing the compound of Formula F4 with a reducing agent to afford a compound of Formula F5: 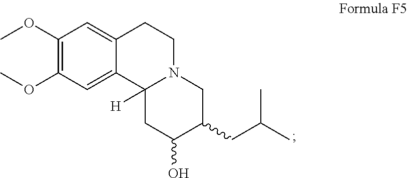 [SMILES: COc1cc2c(cc1OC)C1CC(O)C(CC(C)C)CN1CC2]
d) resolving the compound of Formula F5 with (S)-(+)-camphorsulfonic acid (CSA) to afford a compound of Formula F6-CSA: 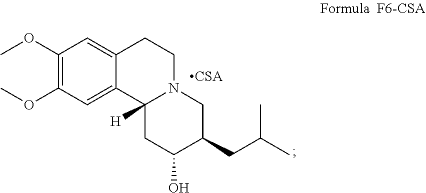 [SMILES: COc1cc2c(cc1OC)[C@H]1C[C@@H](O)[C@H](CC(C)C)CN1CC2]
e) reacting the compound of Formula F6-CSA with a Step e)-base to afford a compound of Formula F6: 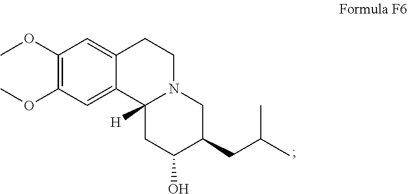 [SMILES: COc1cc2c(cc1OC)[C@H]1C[C@@H](O)[C@H](CC(C)C)CN1CC2]
f) coupling the compound of Formula F6 and a carboxylic acid of Formula F7: 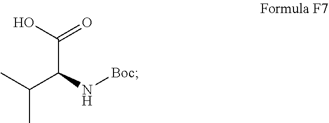
with a coupling reagent to afford a compound of Formula F8: 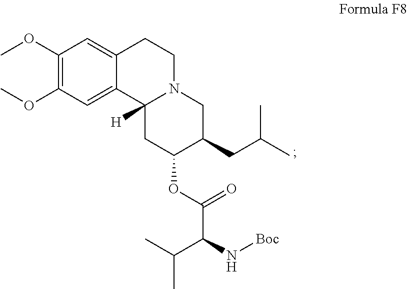 [SMILES: COc1cc2c(cc1OC)[C@H]1C[C@@H](OC(=O)[C@@H](NC(=O)OC(C)(C)C)C(C)C)[C@H](CC(C)C)CN1CC2]
g) deprotecting the compound of Formula F8 with hydrogen chloride to afford a compound of Formula F9-HCl: 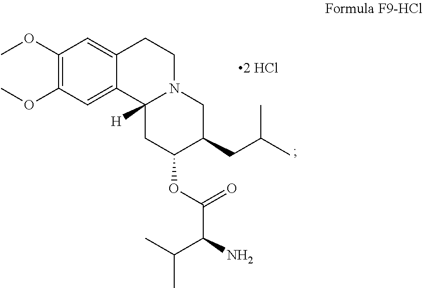 [SMILES: COc1cc2c(cc1OC)[C@H]1C[C@@H](OC(=O)[C@@H](N)C(C)C)[C@H](CC(C)C)CN1CC2]
h) reacting the compound of Formula F9-HCl with a Step h)-base to afford a compound of Formula F9 (free base): 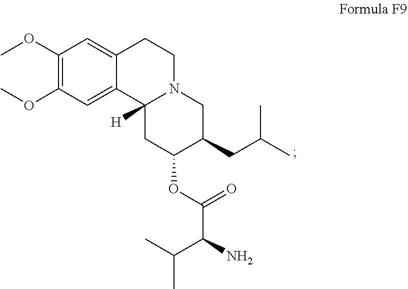 [SMILES: COc1cc2c(cc1OC)[C@H]1C[C@@H](OC(=O)[C@@H](N)C(C)C)[C@H](CC(C)C)CN1CC2]
and
i) reacting the compound of Formula F9 with p-toluenesulfonic acid to afford the compound of Formula I.

2. The process according to claim 1, wherein reacting the compound of Formula F1 with a Step a)-base is carried out in the presence of a Step a)-solvent.

3. The process according to claim 1, wherein reacting the compound of Formula F1 with a Step a)-base is carried out in the presence of a Step a)-solvent comprising methyl tert-butyl ether (MTBE).

4. The process according to claim 1, wherein reacting the compound of Formula F1 with a Step a)-base is carried out in the presence of methyl tert-butyl ether (MTBE).

5. The process according to claim 1, wherein reacting the compound of Formula F1 with a Step a)-base is carried out in the presence of water and methyl tert-butyl ether (MTBE).

6. The process according to any one of claims 1 to 5, wherein the Step a)-base is potassium hydroxide.

7. The process according to any one of claims 1 to 5, wherein the Step a)-base is aqueous potassium hydroxide.

8. The process according to any one of claims 1 to 7, wherein reacting the compound of Formula F1 with a Step a)-base is carried out at a pH of about 10 to about 12.

9. The process according to any one of claims 1 to 7, wherein reacting the compound of Formula F1 with a Step a)-base is carried out at a pH of about 11.

10. The process according to any one of claims 1 to 9, wherein the molar ratio of sodium iodide to the compound of Formula F3 is about 0.35:1 to 0.45:1.

11. The process according to any one of claims 1 to 10, wherein cyclizing the compound of Formula F2 with a compound of Formula F3 in the presence of sodium iodide in Step b) is carried out in a cyclizing-step solvent.

12. The process according to any one of claims 1 to 10, wherein cyclizing the compound of Formula F2 with a compound of Formula F3 in the presence of sodium iodide in Step b) is carried out in a cyclizing-step solvent comprising isopropanol (IPA) and water.

13. The process according to any one of claims 1 to 10, wherein cyclizing the compound of Formula F2 with a compound of Formula F3 in the presence of sodium iodide in Step b) is carried out in isopropanol (IPA) and water.

14. The process according to any one of claims 1 to 13, wherein reducing the compound of Formula F4 with a reducing agent in Step c) is carried out in a reducing-step solvent.

15. The process according to any one of claims 1 to 13, wherein reducing the compound of Formula F4 with a reducing agent in Step c) is carried out in a reducing-step solvent comprising methyl tert-butyl ether (MTBE) and methanol.

16. The process according to any one of claims 1 to 13, wherein reducing the compound of Formula F4 with a reducing agent in Step c) is carried out in methyl tert-butyl ether (MTBE), acetic acid, and methanol.

17. The process according to any one of claims 1 to 16, wherein the reducing agent in Step c) is sodium borohydride.

18. The process according to any one of claims 1 to 17, wherein resolving the compound of Formula F5 with (S)-(+)-camphorsulfonic acid (CSA) in Step d) is carried out in a resolving-step solvent.

19. The process according to any one of claims 1 to 17, wherein resolving the compound of Formula F5 with (S)-(+)-camphorsulfonic acid (CSA) in Step d) is carried out in a resolving-step solvent comprising ethanol and water.

20. The process according to any one of claims 1 to 17, wherein resolving the compound of Formula F5 with (S)-(+)-camphorsulfonic acid (CSA) in Step d) is carried out in ethanol and water.

21. The process according to any one of claims 1 to 20, wherein the Step e)-base is sodium hydroxide.

22. The process according to any one of claims 1 to 20, wherein the Step e)-base is aqueous sodium hydroxide.

23. The process according to any one of claims 1 to 20, wherein the Step e)-base is potassium hydroxide.

24. The process according to any one of claims 1 to 20, wherein the Step e)-base is aqueous potassium hydroxide.

25. The process according to any one of claims 1 to 24, wherein reacting the compound of Formula F6-CSA with a Step e)-base is carried out in the presence of a Step e)-solvent.

26. The process according to any one of claims 1 to 24, wherein reacting the compound of Formula F6-CSA with a Step e)-base is carried out in the presence of a Step e)-solvent comprising dichloromethane.

27. The process according to any one of claims 1 to 24, wherein reacting the compound of Formula F6-CSA with a Step e)-base is carried out in the presence of dichloromethane.

28. The process according to any one of claims 1 to 24, wherein reacting the compound of Formula F6-CSA with a Step e)-base is carried out in the presence of dichloromethane and water.

29. The process according to any one of claims 1 to 24, wherein reacting the compound of Formula F6-CSA with a Step e)-base is carried out in the presence of a Step e)-solvent comprising 2-methyltetrahydrofuran (MeTHF).

30. The process according to any one of claims 1 to 24, wherein reacting the compound of Formula F6-CSA with a Step e)-base is carried out in the presence of 2-methyltetrahydrofuran (MeTHF).

31. The process according to any one of claims 1 to 24, wherein reacting the compound of Formula F6-CSA with a Step e)-base is carried out in the presence of 2-methyltetrahydrofuran (MeTHF) and water.

32. The process according to any one of claims 1 to 31, wherein coupling the compound of Formula F6 and a carboxylic acid of Formula F7 with a coupling reagent in Step f) is carried out in the presence of a coupling-step base.

33. The process according to any one of claims 1 to 31, wherein coupling the compound of Formula F6 and a carboxylic acid of Formula F7 with a coupling reagent in Step f) is carried out in the presence of a coupling-step base comprising 4-dimethylaminopyridine (DMAP).

34. The process according to any one of claims 1 to 31, wherein coupling the compound of Formula F6 and a carboxylic acid of Formula F7 with a coupling reagent in Step f) is carried out in the presence of 4-dimethylaminopyridine (DMAP).

35. The process according to any one of claims 1 to 34, wherein coupling the compound of Formula F6 and a carboxylic acid of Formula F7 with a coupling reagent in Step f) is carried out in the presence of a coupling-step solvent.

36. The process according to any one of claims 1 to 34, wherein coupling the compound of Formula F6 and a carboxylic acid of Formula F7 with a coupling reagent in Step f) is carried out in the presence of a coupling-step solvent comprising dichloromethane.

37. The process according to any one of claims 1 to 34, wherein coupling the compound of Formula F6 and a carboxylic acid of Formula F7 with a coupling reagent in Step f) is carried out in the presence of dichloromethane.

38. The process according to any one of claims 1 to 34, wherein coupling the compound of Formula F6 and a carboxylic acid of Formula F7 with a coupling reagent in Step f) is carried out in the presence of a coupling-step solvent comprising 2-methyltetrahydrofuran (MeTHF).

39. The process according to any one of claims 1 to 34, wherein coupling the compound of Formula F6 and a carboxylic acid of Formula F7 with a coupling reagent in Step f) is carried out in the presence of 2-methyltetrahydrofuran (MeTHF).

40. The process according to any one of claims 1 to 39, wherein the coupling reagent is N-(3-dimethylaminopropyl)-N′-ethylcarbodiimide hydrochloride (EDC. HCl).

41. The process according to any one of claims 1 to 39, wherein the coupling reagent is N-(3-dimethylaminopropyl)-N′-ethylcarbodiimide (EDCI).

42. The process according to any one of claims 1 to 41, wherein the hydrogen chloride in Step g) is a hydrogen chloride dioxane mixture.

43. The process according to any one of claims 1 to 41, wherein the hydrogen chloride in Step g) is a hydrogen chloride isopropanol (IPA) mixture.

44. The process according to any one of claims 1 to 41, wherein the hydrogen chloride in Step g) is substantially anhydrous.

45. The process according to any one of claims 1 to 44, wherein deprotecting the compound of Formula F8 with hydrogen chloride in Step g) is carried out in a deprotecting-step solvent.

46. The process according to any one of claims 1 to 44, wherein deprotecting the compound of Formula F8 with hydrogen chloride in Step g) is carried out in a deprotecting-step solvent comprising dichloromethane.

47. The process according to any one of claims 1 to 44, wherein deprotecting the compound of Formula F8 with hydrogen chloride in Step g) is carried out in dichloromethane.

48. The process according to any one of claims 1 to 44, wherein deprotecting the compound of Formula F8 with hydrogen chloride in Step g) is carried out in a deprotecting-step solvent comprising 2-methyltetrahydrofuran (MeTHF).

49. The process according to any one of claims 1 to 44, wherein deprotecting the compound of Formula F8 with hydrogen chloride in Step g) is carried out in a deprotecting-step solvent comprising 2-methyltetrahydrofuran (MeTHF) and ethyl acetate (EtOAc).

50. The process according to any one of claims 1 to 44, wherein deprotecting the compound of Formula F8 with hydrogen chloride in Step g) is carried out in 2-methyltetrahydrofuran (MeTHF).

51. The process according to any one of claims 1 to 44, wherein deprotecting the compound of Formula F8 with hydrogen chloride in Step g) is carried out in 2-methyltetrahydrofuran (MeTHF) and ethyl acetate (EtOAc).

52. The process according to any one of claims 1 to 51, wherein after Step g) and before Step h) the process further comprises the steps of:
1) reacting the compound of Formula F9-HCl with aqueous sodium bicarbonate in the presence of a solvent comprising dichloromethane to afford a compound of Formula F9 (free base); and
2) reacting the compound of Formula F9 (free base) in a solvent comprising acetonitrile with a hydrogen chloride isopropanol mixture to afford the compound of Formula F9-HCl.

53. The process according to any one of claims 1 to 52, wherein the Step h)-base is sodium bicarbonate.

54. The process according to any one of claims 1 to 52, wherein the Step h)-base is aqueous sodium bicarbonate.

55. The process according to any one of claims 1 to 54, wherein reacting the compound of Formula F9-HCl with a Step h)-base is carried out in the presence of a Step h)-solvent.

56. The process according to any one of claims 1 to 54, wherein reacting the compound of Formula F9-HCl with a Step h)-base is carried out in the presence of a Step h)-solvent comprising dichloromethane.

57. The process according to any one of claims 1 to 54, wherein reacting the compound of Formula F9-HCl with a Step h)-base is carried out in the presence of dichloromethane.

58. The process according to any one of claims 1 to 54, wherein reacting the compound of Formula F9-HCl with a Step h)-base is carried out in the presence of dichloromethane and water.

59. The process according to any one of claims 1 to 54, wherein reacting the compound of Formula F9-HCl with a Step h)-base is carried out in the presence of a Step h)-solvent comprising 2-methyltetrahydrofuran (MeTHF).

60. The process according to any one of claims 1 to 54, wherein reacting the compound of Formula F9-HCl with a Step h)-base is carried out in the presence of a Step h)-solvent comprising ethyl acetate (EtOAc).

61. The process according to any one of claims 1 to 54, wherein reacting the compound of Formula F9-HCl with a Step h)-base is carried out in the presence of a Step h)-solvent comprising 2-methyltetrahydrofuran (MeTHF) and ethyl acetate (EtOAc).

62. The process according to any one of claims 1 to 54, wherein reacting the compound of Formula F9-HCl with a Step h)-base is carried out in 2-methyltetrahydrofuran (MeTHF) and ethyl acetate (EtOAc).

63. The process according to any one of claims 1 to 62, wherein reacting the compound of Formula F9 with p-toluenesulfonic acid in Step i) is carried out in a solvent comprising acetonitrile.

64. The process according to any one of claims 1 to 62, wherein reacting the compound of Formula F9 with p-toluenesulfonic acid in Step i) is carried out in acetonitrile.

65. A process for preparing a compound of Formula I: 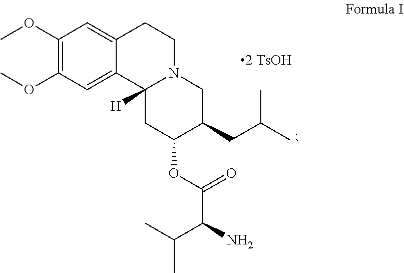 [SMILES: COc1cc2c(cc1OC)[C@H]1C[C@@H](OC(=O)[C@@H](N)C(C)C)[C@H](CC(C)C)CN1CC2]
comprising the steps of:
a) reacting a compound of Formula F1: 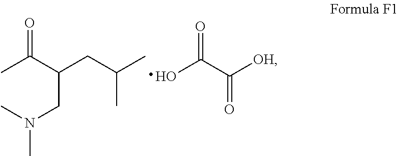
with aqueous potassium hydroxide in the presence of methyl tert-butyl ether (MTBE) to afford a compound of Formula F2: 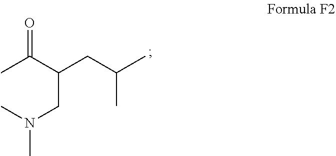
b) cyclizing the compound of Formula F2 with a compound of Formula F3: 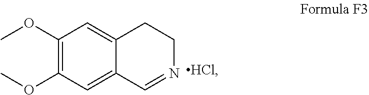
in the presence of sodium iodide, isopropanol (IPA), and water to afford a compound of Formula F4: 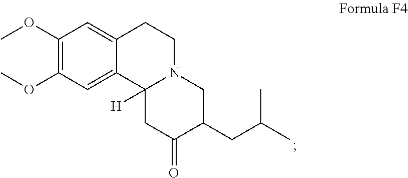 [SMILES: COc1cc2c(cc1OC)C1CC(=O)C(CC(C)C)CN1CC2]
c) reducing the compound of Formula F4 with sodium borohydride in the presence of methyl tert-butyl ether (MTBE), acetic acid, and methanol to afford a compound of Formula F5: 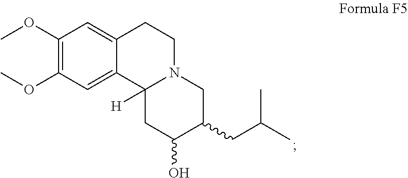 [SMILES: COc1cc2c(cc1OC)C1CC(O)C(CC(C)C)CN1CC2]
d) resolving the compound of Formula F5 with (S)-(+)-camphorsulfonic acid (CSA) in ethanol and water to afford a compound of Formula F6-CSA: 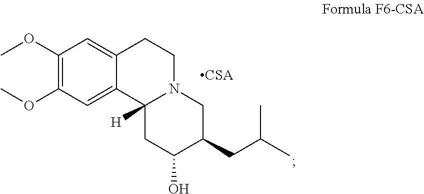 [SMILES: COc1cc2c(cc1OC)[C@H]1C[C@@H](O)[C@H](CC(C)C)CN1CC2]
e) reacting the compound of Formula F6-CSA with aqueous sodium hydroxide in dichloromethane to afford a compound of Formula F6: 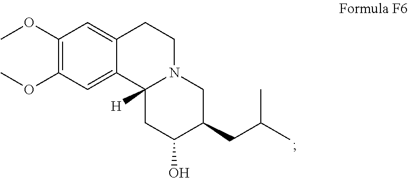 [SMILES: COc1cc2c(cc1OC)[C@H]1C[C@@H](O)[C@H](CC(C)C)CN1CC2]
f) coupling the compound of Formula F6 and a carboxylic acid of Formula F7: 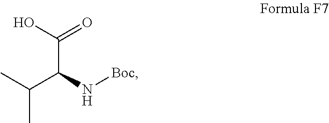
with N-(3-dimethylaminopropyl)-N′-ethylcarbodiimide hydrochloride (EDC·HCl) in the presence of 4-dimethylaminopyridine (DMAP) and dichloromethane to afford a compound of Formula F8: 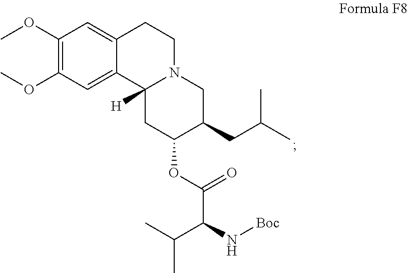 [SMILES: COc1cc2c(cc1OC)[C@H]1C[C@@H](OC(=O)[C@@H](NC(=O)OC(C)(C)C)C(C)C)[C@H](CC(C)C)CN1CC2]
g) deprotecting the compound of Formula F8 with a mixture of hydrogen chloride and dioxane in the presence of dichloromethane to afford a compound of Formula F9-HCl: 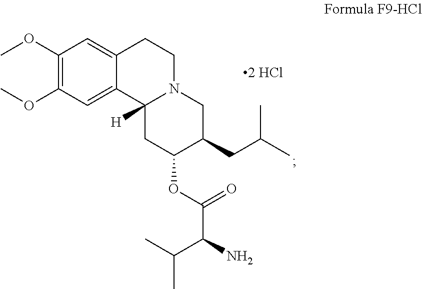 [SMILES: COc1cc2c(cc1OC)[C@H]1C[C@@H](OC(=O)[C@@H](N)C(C)C)[C@H](CC(C)C)CN1CC2]
h) reacting the compound of Formula F9-HCl with aqueous sodium bicarbonate in the presence of dichloromethane to afford a compound of Formula F9 (free base): 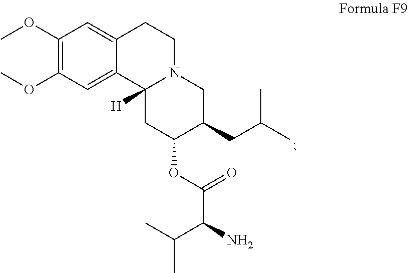 [SMILES: COc1cc2c(cc1OC)[C@H]1C[C@@H](OC(=O)[C@@H](N)C(C)C)[C@H](CC(C)C)CN1CC2]
and
i) reacting the compound of Formula F9 with p-toluenesulfonic acid in the presence of acetonitrile to afford the compound of Formula I.

66. The process according to claim 65, wherein after Step g) and before Step h) the process further comprises the steps of:
1) reacting the compound of Formula F9-HCl with aqueous sodium bicarbonate in the presence of a solvent comprising dichloromethane to afford a compound of Formula F9 (free base): 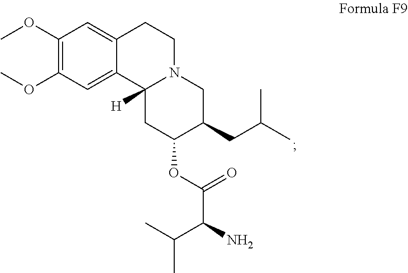 [SMILES: COc1cc2c(cc1OC)[C@H]1C[C@@H](OC(=O)[C@@H](N)C(C)C)[C@H](CC(C)C)CN1CC2]
and
2) reacting the compound of Formula F9 (free base) in a solvent comprising acetonitrile with a hydrogen chloride isopropanol mixture to afford the compound of Formula F9-HCl.

67. A process for preparing a compound of Formula I: 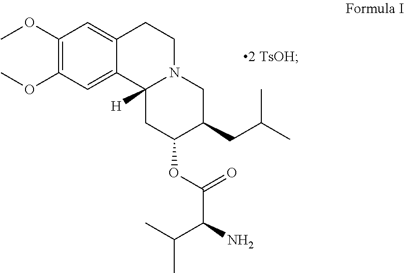 [SMILES: COc1cc2c(cc1OC)[C@H]1C[C@@H](OC(=O)[C@@H](N)C(C)C)[C@H](CC(C)C)CN1CC2]
comprising the steps of:
a) reacting a compound of Formula F1: 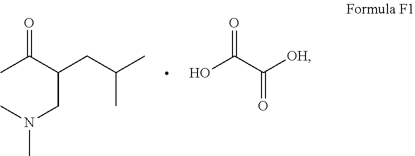
with aqueous potassium hydroxide in the presence of methyl tert-butyl ether (MTBE) to afford a compound of Formula F2: 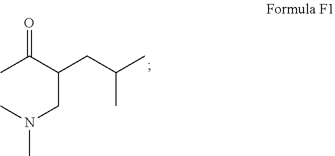
b) cyclizing the compound of Formula F2 with a compound of Formula F3: 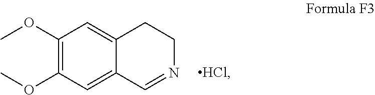
in the presence of sodium iodide, isopropanol (IPA), and water to afford a compound of Formula F4: 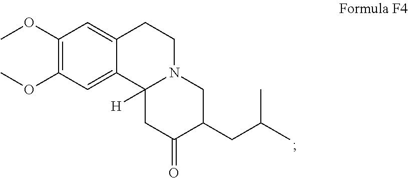 [SMILES: COc1cc2c(cc1OC)C1CC(=O)C(CC(C)C)CN1CC2]
c) reducing the compound of Formula F4 with sodium borohydride in the presence of methyl tert-butyl ether (MTBE), acetic acid, and methanol to afford a compound of Formula F5: 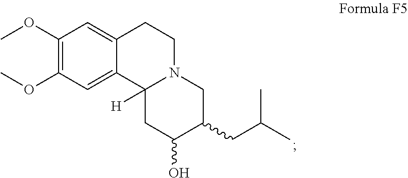 [SMILES: COc1cc2c(cc1OC)C1CC(O)C(CC(C)C)CN1CC2]
d) resolving the compound of Formula F5 with (S)-(+)-camphorsulfonic acid (CSA) in ethanol and water to afford a compound of Formula F6-CSA: 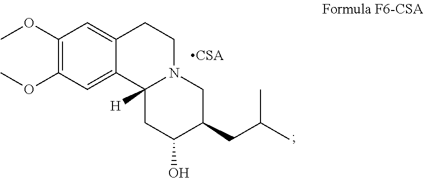 [SMILES: COc1cc2c(cc1OC)[C@H]1C[C@@H](O)[C@H](CC(C)C)CN1CC2]
e) reacting the compound of Formula F6-CSA with aqueous sodium hydroxide in dichloromethane to afford a compound of Formula F6: 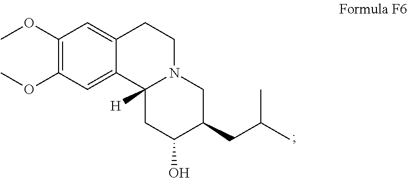 [SMILES: COc1cc2c(cc1OC)[C@H]1C[C@@H](O)[C@H](CC(C)C)CN1CC2]
f) coupling the compound of Formula F6 and a carboxylic acid of Formula F7: 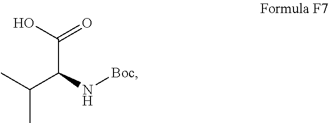
with N-(3-dimethylaminopropyl)-N′-ethylcarbodiimide hydrochloride (EDC·HCl) in the presence of 4-dimethylaminopyridine (DMAP) and dichloromethane to afford a compound of Formula F8: 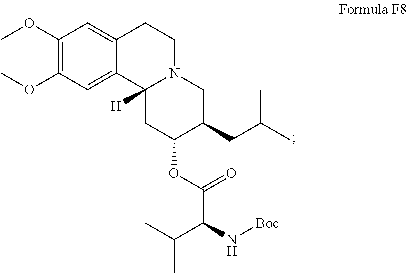 [SMILES: COc1cc2c(cc1OC)[C@H]1C[C@@H](OC(=O)[C@@H](NC(=O)OC(C)(C)C)C(C)C)[C@H](CC(C)C)CN1CC2]
g) deprotecting the compound of Formula F8 with a mixture of hydrogen chloride and dioxane in the presence of dichloromethane to afford a compound of Formula F9-HCl: 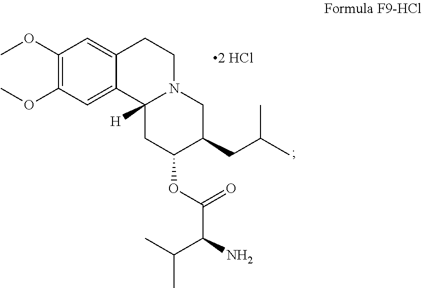 [SMILES: COc1cc2c(cc1OC)[C@H]1C[C@@H](OC(=O)[C@@H](N)C(C)C)[C@H](CC(C)C)CN1CC2]
reacting the compound of Formula F9-HCl with aqueous sodium bicarbonate in the presence of a solvent comprising dichloromethane to afford a compound of Formula F9 (free base): 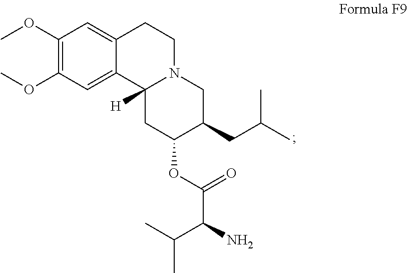 [SMILES: COc1cc2c(cc1OC)[C@H]1C[C@@H](OC(=O)[C@@H](N)C(C)C)[C@H](CC(C)C)CN1CC2]
reacting the compound of Formula F9 (free base) in a solvent comprising acetonitrile with a hydrogen chloride isopropanol mixture to afford the compound of Formula F9-HCl;
h) reacting the compound of Formula F9-HCl with aqueous sodium bicarbonate in the presence of dichloromethane to afford a compound of Formula F9 (free base); and
i) reacting the compound of Formula F9 with p-toluenesulfonic acid in the presence of acetonitrile to afford the compound of Formula I.

68. A process for preparing a compound of Formula I: 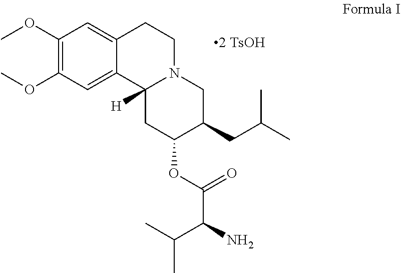 [SMILES: COc1cc2c(cc1OC)[C@H]1C[C@@H](OC(=O)[C@@H](N)C(C)C)[C@H](CC(C)C)CN1CC2]
comprising the steps of:
a) reacting a compound of Formula F1: 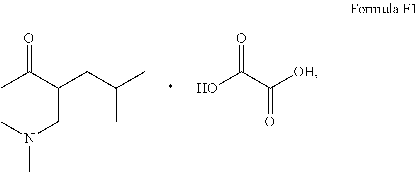
with aqueous potassium hydroxide in the presence of methyl tert-butyl ether (MTBE) to afford a compound of Formula F2: 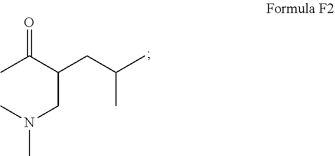
b) cyclizing the compound of Formula F2 with a compound of Formula F3: 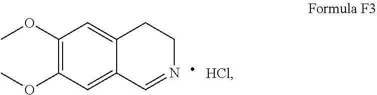
in the presence of sodium iodide, isopropanol (IPA), and water to afford a compound of Formula F4: 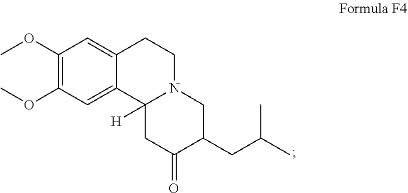 [SMILES: COc1cc2c(cc1OC)C1CC(=O)C(CC(C)C)CN1CC2]
c) reducing the compound of Formula F4 with sodium borohydride in the presence of methyl tert-butyl ether (MTBE), acetic acid, and methanol to afford a compound of Formula F1: 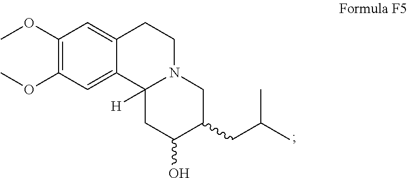 [SMILES: COc1cc2c(cc1OC)C1CC(O)C(CC(C)C)CN1CC2]
d) resolving the compound of Formula F5 with (S)-(+)-camphorsulfonic acid (CSA) in ethanol and water to afford a compound of Formula F6-CSA: 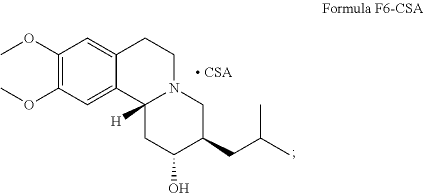 [SMILES: COc1cc2c(cc1OC)[C@H]1C[C@@H](O)[C@H](CC(C)C)CN1CC2]
e) reacting the compound of Formula F6-CSA with aqueous potassium hydroxide in 2-methyltetrahydrofuran (MeTHF) to afford a compound of Formula F6: 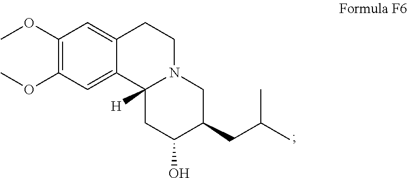 [SMILES: COc1cc2c(cc1OC)[C@H]1C[C@@H](O)[C@H](CC(C)C)CN1CC2]
f) coupling the compound of Formula F6 and a carboxylic acid of Formula F7: 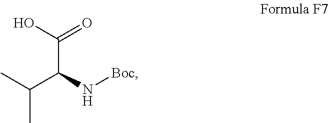
with N-(3-dimethylaminopropyl)-N′-ethylcarbodiimide (EDCI) in the presence of 4-dimethylaminopyridine (DMAP) and 2-methyltetrahydrofuran (MeTHF) to afford a compound of Formula F8: 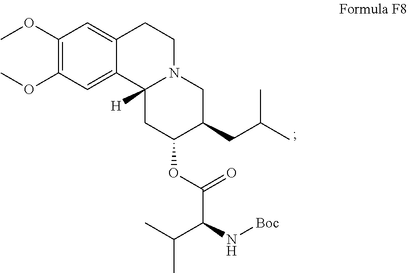 [SMILES: COc1cc2c(cc1OC)[C@H]1C[C@@H](OC(=O)[C@@H](NC(=O)OC(C)(C)C)C(C)C)[C@H](CC(C)C)CN1CC2]
g) deprotecting the compound of Formula F8 with a mixture of hydrogen chloride and isopropanol in the presence of 2-methyltetrahydrofuran (MeTHF) and ethyl acetate (EtOAc) to afford a compound of Formula F9-HCl: 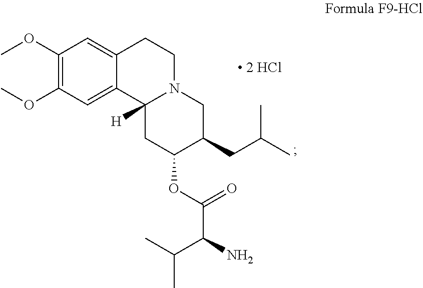 [SMILES: COc1cc2c(cc1OC)[C@H]1C[C@@H](OC(=O)[C@@H](N)C(C)C)[C@H](CC(C)C)CN1CC2]
h) reacting the compound of Formula F9-HCl with aqueous sodium bicarbonate in the presence of 2-methyltetrahydrofuran (MeTHF) and ethyl acetate (EtOAc) to afford a compound of Formula F9 (free base): 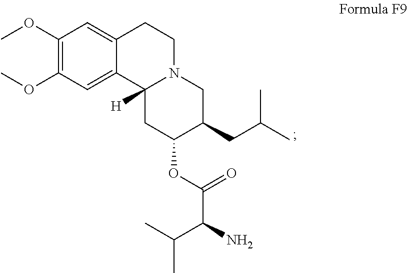 [SMILES: COc1cc2c(cc1OC)[C@H]1C[C@@H](OC(=O)[C@@H](N)C(C)C)[C@H](CC(C)C)CN1CC2]
and i) reacting the compound of Formula F9 with p-toluenesulfonic acid in the presence of acetonitrile to afford the compound of Formula I.

69. A process for preparing a compound of Formula I: 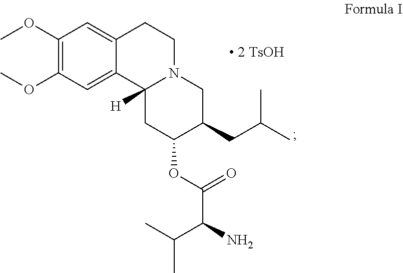 [SMILES: COc1cc2c(cc1OC)[C@H]1C[C@@H](OC(=O)[C@@H](N)C(C)C)[C@H](CC(C)C)CN1CC2]
comprising the steps of:
a) reacting a compound of Formula F1: 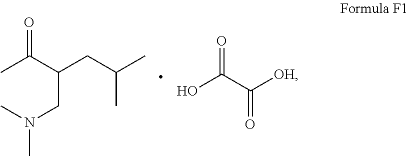
with aqueous potassium hydroxide in the presence of methyl tert-butyl ether (MTBE) to afford a compound of Formula F2: 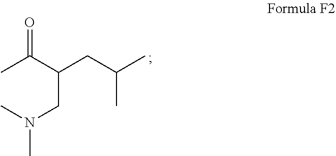
b) cyclizing the compound of Formula F2 with a compound of Formula F3: 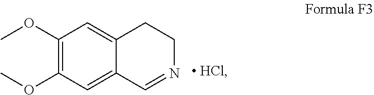
in the presence of sodium iodide, isopropanol (IPA), and water to afford a compound of Formula F4: 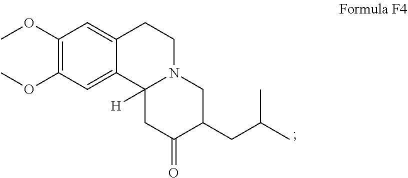 [SMILES: COc1cc2c(cc1OC)C1CC(=O)C(CC(C)C)CN1CC2]
c) reducing the compound of Formula F4 with sodium borohydride in the presence of methyl tert-butyl ether (MTBE), acetic acid, and methanol to afford a compound of Formula F5: 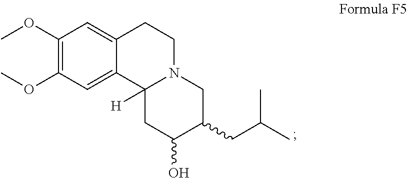 [SMILES: COc1cc2c(cc1OC)C1CC(O)C(CC(C)C)CN1CC2]
d) resolving the compound of Formula F5 with (S)-(+)-camphorsulfonic acid (CSA) in ethanol and water to afford a compound of Formula F6-CSA: 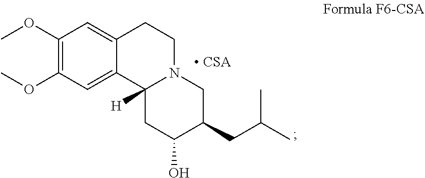 [SMILES: COc1cc2c(cc1OC)[C@H]1C[C@@H](O)[C@H](CC(C)C)CN1CC2]
e) reacting the compound of Formula F6-CSA with aqueous sodium hydroxide in dichloromethane to afford a compound of Formula F6: 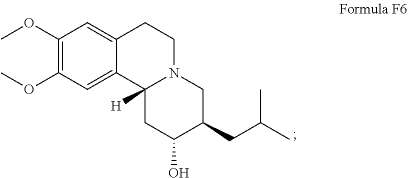 [SMILES: COc1cc2c(cc1OC)[C@H]1C[C@@H](O)[C@H](CC(C)C)CN1CC2]
f) coupling the compound of Formula F6 and a carboxylic acid of Formula F7: 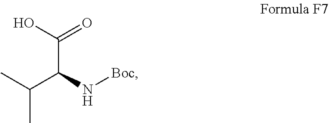
with N-(3-dimethylaminopropyl)-N′-ethylcarbodiimide hydrochloride (EDC·HCl) in the presence of 4-dimethylaminopyridine (DMAP) and dichloromethane to afford a compound of Formula F8: 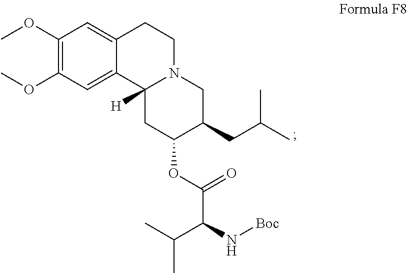 [SMILES: COc1cc2c(cc1OC)[C@H]1C[C@@H](OC(=O)[C@@H](NC(=O)OC(C)(C)C)C(C)C)[C@H](CC(C)C)CN1CC2]
g) deprotecting the compound of Formula F8 with a mixture of hydrogen chloride and dioxane in the presence of dichloromethane to afford a compound of Formula F9-HCl: 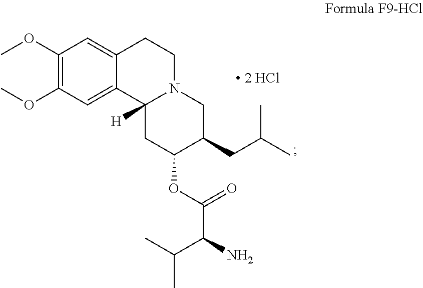 [SMILES: COc1cc2c(cc1OC)[C@H]1C[C@@H](OC(=O)[C@@H](N)C(C)C)[C@H](CC(C)C)CN1CC2]
and
h) reacting the compound of Formula F9-HCl with p-toluenesulfonic acid in a solvent comprising ethyl acetate (EtOAc) to afford the compound of Formula I.

70. The process according to claim 68 or 69, wherein after Step g) and before Step h) the process further comprises the steps of:
1) reacting the compound of Formula F9-HCl with aqueous sodium bicarbonate in the presence of a solvent comprising dichloromethane to afford a compound of Formula F9 (free base): 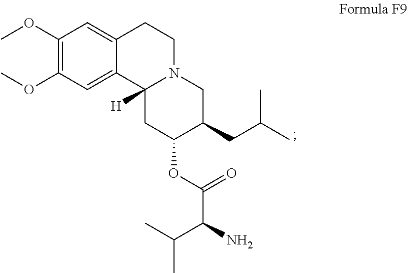 [SMILES: COc1cc2c(cc1OC)[C@H]1C[C@@H](OC(=O)[C@@H](N)C(C)C)[C@H](CC(C)C)CN1CC2]
and
2) reacting the compound of Formula F9 (free base) in a solvent comprising acetonitrile with a hydrogen chloride isopropanol mixture to afford the compound of Formula F9-HCl.

71. The process according to any one of claims 1 to 70, wherein the compound of Formula I is crystalline.

72. The process according to any one of claims 1 to 70, wherein the compound of Formula I is crystalline form I.

73. A process for preparing a pharmaceutical composition comprising: preparing a compound of Formula I according to any one of claims 1 to 72, and formulating the compound of Formula I with a pharmaceutically acceptable carrier and/or diluent.

74. The process for preparing a pharmaceutical composition of claim 73, wherein the pharmaceutically acceptable carrier and/or diluent comprises silicified microcrystalline cellulose: isomalt: hydroxypropyl methylcellulose: partially pregelatinized maize starch:
and magnesium stearate.

75. A process for preparing a unit dosage form comprising: preparing a compound of Formula I according to any one of claims 1 to 74, and formulating the compound of Formula I with a pharmaceutically acceptable carrier and/or diluent.

76. The process for preparing a unit dosage form of claim 75, wherein the pharmaceutically acceptable carrier and/or diluent comprises silicified microcrystalline cellulose; isomalt:
hydroxypropyl methylcellulose: partially pregelatinized maize starch; and magnesium stearate.

77. The process for preparing a unit dosage form of claim 75 or 76, wherein the compound of Formula I in the unit dosage form is present in an amount ranging from about 20 mg to 160 mg as measured as the free base.

78. The process for preparing a unit dosage form of claim 75 or 76, wherein the compound of Formula I in the unit dosage form is present in an amount of 20 mg, 40 mg, 60 mg, 80 mg, or 100 mg as measured as the free base.

79. The process for preparing a unit dosage form according to any one of claims 75 to 78, wherein the unit dosage form is suitable for oral administration.

80. The process for preparing a unit dosage form according to any one of claims 75 to 79, wherein the unit dosage form is formulated for a once daily dosing.

81. The process for preparing a unit dosage form according to any one of claims 75 to 80, wherein the unit dosage form is in a capsule form.

82. The process for preparing a unit dosage form of claim 81, wherein the capsule is size 1 or smaller.

83. The process for preparing a unit dosage form of claim 81, wherein the capsule is size 1, 2, or 3.

84. A pharmaceutical composition prepared by the process according to claim 73 or 74.

85. A unit dosage form prepared by the process according to any one of claims 75 to 83.

86. A method for inhibiting monoamine transporter isoform 2 (VMAT2) in a patient in need thereof, comprising administering to the patient a therapeutically effective amount of a pharmaceutical composition according to claim 84 or a unit dosage form according to claim 85.

87. A method of treating a neurological or psychiatric disease or disorder in a patient in need thereof, comprising administering to the patient a therapeutically effective amount of a pharmaceutical composition according to claim 84 or a unit dosage form according to claim 85.

88. A method of treating a hyperkinetic disorder in a patient in need thereof, comprising administering to the patient a therapeutically effective amount of a pharmaceutical composition according to claim 84 or a unit dosage form according to claim 85.

89. The use of a pharmaceutical composition according to claim 84 or a unit dosage form according to claim 85 for the manufacture of a medicament for inhibiting monoamine transporter isoform 2 (VMAT2) in a patient in need thereof.

90. The use of a pharmaceutical composition according to claim 84 or a unit dosage form according to claim 85 for the manufacture of a medicament for treating a neurological or psychiatric disease or disorder in a patient in need thereof.

91. The use of a pharmaceutical composition according to claim 84 or a unit dosage form according to claim 85 for the manufacture of a medicament for treating a hyperkinetic disorder in a patient in need thereof.
